(19) United States
(12) Patent Application Publication
(10) Pub. No.: US 2016/0046954 A1
(43) Pub. Date: Feb. 18, 2016

(54) METHODS TO IMPROVE CROPS THROUGH INCREASED ACCUMULATION OF METHIONINE

(71) Applicant: PLANT SENSORY SYSTEMS, LLC, Baltimore, MD (US)

(72) Inventors: Michelle B. PRICE, Baltimore, MD (US); Frank J. TURANO, Baltimore, MD (US); Kathleen A. TURANO, Baltimore, MD (US)

(73) Assignee: PLANT SENSORY SYSTEMS, LLC, Baltimore, MD (US)

(21) Appl. No.: 14/462,350

(22) Filed: Aug. 18, 2014

(51) Int. Cl. C12N 15/82; A23K 1/14; C12P 13/12

(52) U.S. Cl. CPC C12N 15/8253; C12P 13/12; A23K 1/14

(57) ABSTRACT

The present invention describes an alternative approach to increase methionine (Met) production in eukaryotes, namely by the insertion of components of a sulfur-metabolic pathway in organisms where the pathway does not exist or has not clearly been identified. The invention describes methods for the use of polynucleotides that encode functional cysteine dioxygenase (CDO) alone or CDO and sulfinoalanine decarboxylase (SAD) polypeptides in plants to increase Met production. The preferred embodiment of the invention is in plants but other organisms may be used. Changes in Met availability will improve nutritional value of the crop.

### SEQUENCE SUBMISSION

The present application is being filed along with a Sequence Listing in electronic format. The Sequence Listing is entitled 3834-114SequenceListing.txt, created on 6 Aug. 2014 and is 19 kb in size. The information in the electronic format of the Sequence Listing is incorporated herein by reference in their entirety.

### FIELD OF THE INVENTION

The present invention is in the field of recombinant production of methionine (Met).

### BACKGROUND OF THE INVENTION

The present invention relates to methods and materials for production of Met in cells and living organisms. More particularly, the invention relates to genetic transformation of organisms, preferably plants, with genes that encode proteins that when present result in increased levels of the sulfur-containing amino acid, Met. The invention also relates to methods to use the plant or plant organs that contain the invention to produce food, animal feed, aquafeed, food supplement, animal-feed supplement, dietary supplement, or health supplement.

The publications and other materials used herein to illuminate the background of the invention or provide additional details respecting the practice, are incorporated by reference, and for convenience are respectively grouped in the Bibliography.

Met is an essential amino acid that cannot be synthesized by non-ruminant animals and must be consumed in their diet.¹ Met is deficient in several grains and other major food staples, such as soybean², potato, and cassava. Over the last 30 years plant breeding and biotech research programs have focused on increasing the Met content in plants and seeds primarily by one of two approaches: increasing the expression of sulfur-rich seed storage proteins^{3, 4} or deregulating Met biosynthesis.⁵⁻¹³ Both approaches have had limited success.¹⁴ Sulfur-rich seed storage proteins do not accumulate to high levels in vegetative tissues^{15, 16} and some sulfur-rich seed storage proteins are potential allergens.¹⁷ Due to the problems associated with expressing sulfur-rich seed storage proteins in plants, recent research efforts to increase Met in plants have focused on the deregulation of the Met biosynthetic pathway. These efforts have focused on modifications of genes and corresponding enzymes that are considered to be rate-limiting steps in the Met biosynthetic and catabolic pathways.¹⁴ The approach has been to over-express or under-express key enzymes in the pathway, namely cystathionine-gamma-synthase (CGS) and threonine synthase (TS) or enzymes that control Met turnover or catabolism. Although these approaches have been shown to increase Met levels in tissue, they have for the most part resulted in plant growth abnormalities.

Sulfur is required for Met biosynthesis.¹⁸⁻²¹ Sulfur is taken up by the plant as sulfate through the roots by transporter proteins.²¹ Most of the sulfur in the form of sulfate is transferred throughout the plant by distinct sulfate transporters or in the form of other sulfur-containing compounds. Once inside the cell sulfate is reduced to sulfide through a series of enzymatic reactions^{22, 23} before it is assimilated into the amino acid cysteine (Cys). Cys biosynthesis occurs in three different locations in the cell: cytosol, plastids, and mitochondria.²⁴⁻²⁶ The enzyme serine acetyltransferase (SAT) controls the production of O-acetylserine (OAS) and the enzyme and end-product in turn control the enzymes involved in sulfate reduction and Cys biosynthesis²⁷⁻³¹ Cys is the source of sulfur for the amino acid, Met.

The compound O-phosphohomoserine is a critical metabolite in Met and threonine metabolism. O-phosphohomoserine is utilized for Met and threonine biosynthesis by the enzymes CGS and TS, respectively.³² Efforts to increase Met levels in plants have focused on the manipulation of the genes for these two enzymes. CGS has been over-expressed in Arabidopsis,³³ tobacco,^{6, 7, 33} potato,^{9, 34} and alfalfa.³⁵ Increased TS activity decreases Met levels in plants and decreased TS, obtained either through mutations¹¹ or by using antisense approaches, has been shown to increase Met accumulation.^{12, 13}

Met catabolism is highly regulated by enzyme S-adenosyl-L-methionine synthetase (SAMS). SAMS converts Met into S-adenosyl-L-methionine (SAM) and SAM functions as a methyl donor.³⁶ SAM is also the precursor of two plant growth regulators, the plant hormone ethylene³⁷⁻⁴⁰ and the polyamines, spermidine and spermine.⁴¹⁻⁴⁴ Suppression of the SAMS gene results in elevated Met levels but abnormal leaf development.⁴⁵

Metabolic Control

The basic concept of modifying the activities of genes that encode rate-limiting enzymes, i.e., to increase desired end products in the pathway, has been heavily investigated with limited success. Recently, challenges to using such an approach has surfaced in the scientific literature.^{46, 47} Introduction of alternative pathways have been shown to be successful in increasing metabolic output perhaps by increasing metabolic flux.^{46, 48} Recent developments to increase sulfur flux through Cys have resulted in an increase in both Cys and Met levels in rice seeds, suggesting the approach may have merit.¹⁴ Another method to increase metabolic flux in a pathway is to add or introduce a novel metabolic pathway or metabolic shortcuts into plants.⁴⁶

The use of the genes, cysteine dioxygenase (CDO) alone or CDO and sulfinoalanine decarboxylase (SAD) (also known as cysteine sulfinic acid decarboxylase) together, have been described to synthetize hypotaurine and taurine in yeast⁴⁹ and plants.⁵⁰ In both cases it was not expected nor predicted that the CDO gene or the CDO and SAD genes would increase Met levels in the cell. Thus there is no obvious reason to those skilled in the art to expect that the addition of a CDO or CDO and SAD would result in increased Met production. This novel method for the use of the CDO gene or the CDO and SAD genes to increase Met is described herein.

### SUMMARY OF THE INVENTION

The present invention relates to methods and compositions to increase sulfur-containing compounds in organisms. More particularly, the invention relates to the use of polynucleotides that encode in plants functional CDO alone or CDO and SAD in combination. The invention provides methods for transforming plants, constructing vector constructs and other nucleic acid molecules for use therein. The transgenic plants will have increased levels of Met for enhanced nutritional quality that can be used as food, feed or supplements in food, aquafeed or animal feed.

In one embodiment of the invention polynucleotides encode a functional CDO gene that encodes a functional enzyme and is used to transform plant cells or to transform plants. In another one embodiment of the invention polynucleotides encode functional CDO and SAD genes that encode functional enzymes and are used to transform plant cells or to transform plants. The inventive methods produce plants that have the advantage of increased levels of sulfur-containing compounds, specifically Met, resulting in plants with increased nutritional value or enhanced plant growth characteristics, survival characteristics and/or tolerance to environmental or other plant stresses. Plants are genetically modified in accordance with the invention to introduce into the plant a polynucleotide that encodes a CDO enzyme alone or polynucleotides that encode CDO and SAD that function in the formation of increased levels of Met in the plant.

### BRIEF DESCRIPTION OF THE FIGURE

FIG. 1 shows an overview of Met biosynthesis and key regulatory enzymes in plant biosynthesis. Sulfur is transported into and throughout the plant as sulfate by specific transporters. Sulfate is reduced into sulfide. Serine acetyltransferase (SAT) controls the synthesis of O-acetyl serine (OAS). OAS and sulfide are used for Cys biosynthesis. Cys and O-phosphohomoserine (OPHS) are used as substrates by cystathionine-gamma-synthase (CGS) to commit metabolites to Met biosynthesis. OPHS can also be used by threonine synthase (TS) for threonine biosynthesis. Met levels are also controlled by its conversion into SAM by the enzyme SAMS. SAM is used in the biosynthesis of ethylene and polyamines and serves as a methyl donor. The insertion of CDO alone or CDO and SAD genes produces the corresponding peptides (light gray oval), which results in the accumulation of Met (broad arrows).

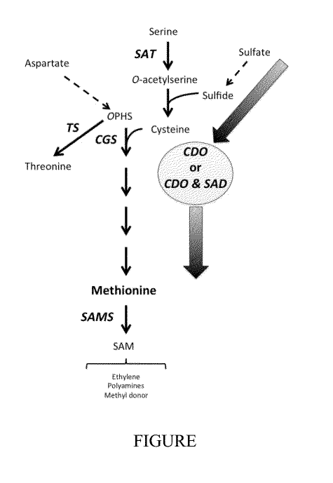

### DETAILED DESCRIPTION OF THE INVENTION

The present invention describes the methods for the synthesis of DNA constructs from polynucleotides and vectors and the methods for making transformed organisms including plants, photosynthetic organisms, microbes, invertebrates, and vertebrates. The present invention is unique in that it describes an alternative approach to increase production of sulfur-containing compounds to increase nutritional value, medical value, growth and development, yield and/or tolerance to biotic and/or abiotic stresses by the insertion of the biosynthetic pathway in organisms where the pathway does not exist or has not clearly been identified. The invention describes methods for the use of polynucleotides that encode functional CDO or CDO and SAD. The preferred embodiment of the invention is in plants but other organisms may be used.

One embodiment of the invention is a method for the production of Met by the following steps:
1. operably link a promoter to the 5′ end of a polynucleotide for a functional chloroplast or plastid transit peptide linked in-frame with a CDO gene with a terminator;
2. insert the polynucleotide construct (from step 1 above) into a vector;
3. transform the vector containing the CDO construct into a plant or plant cell;

One embodiment of the invention is a method for the production of Met by the following steps:
1. operably link a promoter to the 5′ end of the polynucleotide for a functional chloroplast or plastid transit peptide operably linked in-frame with a CDO gene with a terminator;
2. insert the polynucleotide construct (from step 1 above) into a vector;
3. operably link a promoter to the 5′ end of the polynucleotide for the functional chloroplast or plastid transit peptide linked in-frame with a SAD gene with a terminator;
4. insert the SAD polynucleotide construct (from step 3 above) into a vector containing the CDO construct; (from step 2 above) and
5. transform the vector containing the CDO and SAD constructs into a plant or plant cell carrying a CDO construct.

Another embodiment of the invention is a method for the production of Met by the following steps:
1. operably link a promoter to the 5′ end of the polynucleotide for the functional a functional chloroplast or plastid transit peptide linked in-frame with a CDO gene with a terminator;
2. insert the polynucleotide construct (from step 1 above) into a vector;
3. transform the vector containing the CDO construct into a plant or plant cell;
4. operably link a promoter to the 5′ end of the polynucleotide for the functional chloroplast or plastid transit peptide linked in-frame with a SAD gene with a terminator;
5. insert the polynucleotide construct (from step 4 above) into a vector that it is operably linked a terminator;
6. transform the vector containing the SAD construct into a plant or plant cell; and
7. sexually cross a plant (or fuse cells) carrying a CDO construct or one that expresses a functional CDO with a plant (or cells) carrying a SAD construct or one that expresses a functional SAD.

Another embodiment of the invention is a method for the production of Met by the following steps:
1. operably link a promoter to the 5′ end of the polynucleotide for functional chloroplast or plastid transit peptide linked in-frame with a CDO that is linked in-frame with a SAD gene (with no linker) with a operably linked terminator;
2. insert the polynucleotide construct (from step 1 above) into a vector; and
3. transform the vector containing the CDO/SAD construct into a plant or plant cell.

Another embodiment of the invention is a method for the production of Met by the following steps:
1. operably link a promoter to the 5′ end of the polynucleotide for functional chloroplast or plastid transit peptide linked in-frame with a CDO that is linked in-frame with a short polynucleotide (linker) to the 5′ end of the polynucleotide for a functional SAD gene product operably linked a terminator;
2. insert the polynucleotide construct (from step 2 above) into a vector; and
3. transform the vector containing the CDO/linker/SAD construct into a plant or plant cell.

Suitable Polynucleotides for CDO and SAD

Suitable polynucleotides for CDO are provided in SEQ ID NO:1 and SEQ ID NO:2 Other suitable polynucleotides for use in accordance with the invention may be obtained by the identification of polynucleotides that selectively hybridize to the polynucleotides of SEQ ID NO:1 or SEQ ID NO:2 by hybridization under low stringency conditions, moderate stringency conditions, or high stringency conditions. Still other suitable polynucleotides for use in accordance with the invention may be obtained by the identification of polynucleotides that have substantial identity of the nucleic acid of SEQ ID NO:1 or SEQ ID NO:2 when it is used as a reference for sequence comparison or polynucleotides that encode polypeptides that have substantial identity to amino acid sequence of SEQ ID NO:3 or SEQ ID NO:4 when it is used as a reference for sequence comparison. Suitable CDO nucleic acid sequences and corresponding amino acid sequences having a degree of identity or similarity as described herein are identified by GenBank Accession Number in Table 1. The GenBank Accession Number identifies the coding region of the CDO genes. The listed GenBank Accession Numbers are representative and additional nucleic acid sequences can be identified, for example by doing a BLAST search using SEQ ID NO:1, 2, 3 or 4 or any of the listed accession numbers. Thus, it is evident that any CDO gene is contemplated for use in the present invention.

TABLE 1
CDO Nucleic Acid and Amino Acid Sequences

| Nucleic Acid | % | Amino Acid |
|---|---|---|
| Accession No. | Identities | Accession No |
| XM_005901940.1 | 99^{a} | XP_005902002.1 |
| XM_005685043.1 | 98^{a} | XP_005685100.1 |
| XM_005980109.1 | 97^{a} | XP_005980171.1 |
| XM_007192120.1 | 96^{a} | XP_007192182.1 |
| XM_007118375.1 | 95^{a} | XP_007118437.1 |
| XM_006203955.1 | 94^{a} | XP_006204017.1 |
| XM_004467258.1 | 93^{a} | XP_004467315.1 |
| XM_007521965.1 | 92^{a} | XP_007522027.1 |
| XM_004376976.1 | 91^{a} | XP_004377033.1 |
| XM_007954519.1 | 90^{a} | XP_007952710.1 |
| XM_006977659.1 | 89^{a} | XP_006977721.1 |
| XM_002710128.2 | 88^{a} | XP_002710174.1 |
| XM_003761273.1 | 85^{a} | XP_003761321.1 |
| AB220583.1 | 85^{b} | BAE73111.1 |
| XM_008294063.1 | 80^{b} | XP_008292285.1 |
| AB638837.1 | 79^{b} | BAL22276.1 |
| BT082996.1 | 78^{b} | ACQ58703.1 |
| JN216942.1 | 77^{b} | AEM37687.1 |
| XM_002710128.2 | 74^{b} | XP_002710174.1 |
| NM_001141521.2 | 73^{b} | NP_001134993.1 |

^{a}% identities with respect to SEQ ID NO: 1 within coding region
^{b}% identities with respect to SEQ ID NO: 2 within coding region

Suitable polynucleotides for SAD are provided in SEQ ID NO:5 and SEQ ID NO:6. Other suitable polynucleotides for use in accordance with the invention may be obtained by the identification of polynucleotides that selectively hybridize to the polynucleotides of SEQ ID NO:5 or SEQ ID NO:6 by hybridization under low stringency conditions, moderate stringency conditions, or high stringency conditions. Still other suitable polynucleotides for use in accordance with the invention may be obtained by the identification of polynucleotides that have substantial identity of the nucleic acid of SEQ ID NO:5 or SEQ ID NO:6 when it is used as a reference for sequence comparison or polynucleotides that encode polypeptides that have substantial identity to amino acid sequence of SEQ ID NO:7 or SEQ ID NO:8 when it is used as a reference for sequence comparison. Suitable SAD nucleic acid sequences and corresponding amino acid sequences having a degree of identity or similarity as described herein are identified by GenBank Accession Number in Table 2. The GenBank Accession Number identifies the coding region of the SAD genes. The listed GenBank Accession Numbers are representative and additional nucleic acid sequences can be identified, for example by doing a BLAST search using SEQ ID NO:5, 6, 7 or 8 or any of the listed accession numbers. Thus, it is evident that any SAD gene is contemplated for use in the present invention.

TABLE 2
SAD Nucleic Acid and Amino Acid Sequences

| Nucleic Acid | % | Amino Acid |
|---|---|---|
| Accession No. | Identities | Accession No |
| XM_008532994.1 | 99^{a} | XP_008531216.1 |
| XM_004429096.1 | 94^{a} | XP_004429153.1 |
| XM_002923274.1 | 93^{a} | XP_002923320.1 |
| XM_007179802.1 | 93^{a} | XP_007179864.1 |
| XM_004406235.1 | 92^{a} | XP_004406292.1 |
| XM_007081368.1 | 92^{a} | XP_007081430.1 |
| XM_006757018.1 | 91^{a} | XP_006757081.1 |
| XM_001788351.4 | 91^{a} | XP_001788403.2 |
| XM_003952183.1 | 91^{a} | XP_003952232.1 |
| XM_005954400.1 | 91^{a} | XP_005954462.1 |
| XM_004006310.1 | 90^{a} | XP_004006359.1 |
| AB220585.1 | 89^{b} | BAE73113.1 |
| AB638838.1 | 73^{b} | BAL22277.1 |
| XM_007058524.1 | 70^{b} | XP_007058586. |
| XM_006118882.1 | 69^{b} | XP_006118944.1 |

^{a}% identities with respect to SEQ ID NO: 5 within coding region
^{b}% identities with respect to SEQ ID NO: 6 within coding region

Variability and Modification of Sequences

Those of ordinary skill in the art know that organisms of a wide variety of species commonly express and utilize homologous proteins, which include the insertions, substitutions and/or deletions discussed above, and effectively provide similar function. For example, the amino acid sequences for CDO or SAD from zebra fish (Danio rerio) may differ to a certain degree from the amino acid sequences of CDO or SAD in another species and yet have similar functionality with respect to catalytic and regulatory function. Amino acid sequences comprising such variations are included within the scope of the present invention and are considered substantially or sufficiently similar to a reference amino acid sequence. Although it is not intended that the present invention be limited by any theory by which it achieves its advantageous result, it is believed that the identity between amino acid sequences that is necessary to maintain proper functionality is related to maintenance of the tertiary structure of the polypeptide such that specific interactive sequences will be properly located and will have the desired activity, and it is contemplated that a polypeptide including these interactive sequences in proper spatial context will have activity.

Another manner in which similarity may exist between two amino acid sequences is where there is conserved substitution between a given amino acid of one group, such as a non-polar amino acid, an uncharged polar amino acid, a charged polar acidic amino acid, or a charged polar basic amino acid, with an amino acid from the same amino acid group. For example, it is known that the uncharged polar amino acid serine may commonly be substituted with the uncharged polar amino acid threonine in a polypeptide without substantially altering the functionality of the polypeptide. Whether a given substitution will affect the functionality of the enzyme may be determined without undue experimentation using synthetic techniques and screening assays known to one with ordinary skill in the art.

Another embodiment of the invention is a polynucleotide (e.g., a DNA construct) that encodes a protein that functions as a CDO or SAD and selectively hybridizes to either SEQ ID NO:1, SEQ ID NO:2, SEQ ID NO:5 or SEQ ID NO:6, respectively. Selectively hybridizing sequences typically have at least 40% sequence identity, preferably 60-90% sequence identity, and most preferably 100% sequence identity with each other.

Another embodiment of the invention is a polynucleotide that encodes a polypeptide that has substantial identity to the amino acid sequence of SEQ ID NO:3, SEQ ID NO:4, SEQ ID NO:7, or SEQ ID NO:8. Substantial identity of amino acid sequences for these purposes normally means sequence identity of between 50-100%, preferably at least 55%, preferably at least 60%, more preferably at least 70%, 80%, 90%, and most preferably at least 95%.

The process of encoding a specific amino acid sequence may involve DNA sequences having one or more base changes (i.e., insertions, deletions, substitutions) that do not cause a change in the encoded amino acid, or which involve base changes which may alter one or more amino acids, but do not eliminate the functional properties of the polypeptide encoded by the DNA sequence.

It is therefore understood that the invention encompasses more than the specific polynucleotides encoding the proteins described herein. For example, modifications to a sequence, such as deletions, insertions, or substitutions in the sequence, which produce “silent” changes that do not substantially affect the functional properties of the resulting polypeptide are expressly contemplated by the present invention. Furthermore, because of the degeneracy of the genetic code, a large number of functionally identical nucleic acids encode any given protein. For instance, the codons GCA, GCC, GCG and GCU all encode the amino acid alanine. Thus, at every position where an alanine is specified by a codon, the codon can be altered to any of the corresponding codons described without altering the encoded polypeptide. Such nucleic acid variations are “silent variations” and represent one species of conservatively modified variation. Every nucleic acid sequence herein that encodes a polypeptide also describes every possible silent variation of the nucleic acid. One of ordinary skill in the art will recognize that each amino acid has more than one codon, except for Met and tryptophan that ordinarily have the codons AUG and UGG, respectively. It is known by those of ordinary skill in the art, “universal” code is not completely universal. Some mitochondrial and bacterial genomes diverge from the universal code, e.g., some termination codons in the universal code specify amino acids in the mitochondria or bacterial codes. Thus each silent variation of a nucleic acid, which encodes a polypeptide of the present invention, is implicit in each described polypeptide sequence and incorporated in the descriptions of the invention.

One of ordinary skill in the art will recognize that changes in the amino acid sequences, such as individual substitutions, deletions or additions to a nucleic acid, peptide, polypeptide, or protein sequence which alters, adds or deletes a single amino acid or a small percentage of amino acids in the encoded sequence is “sufficiently similar” when the alteration results in the substitution of an amino acid with a chemically similar amino acid. Thus, any number of amino acid residues selected from the group of integers consisting of from 1 to 15 can be so altered. Thus, for example, 1, 2, 3, 4, 5, 7 or 10 alterations can be made. Conservatively modified variants typically provide similar biological activity as the unmodified polypeptide sequence from which they are derived. For example, CDO or SAD activity is generally at least 40%, 50%, 60%, 70%, 80% or 90%, preferably 60-90% of the native protein for the native substrate. Tables of conserved substitution provide lists of functionally similar amino acids.

The following three groups each contain amino acids that are conserved substitutions for one another: (1) Alanine (A), Serine (S), Threonine (T); (2) Aspartic acid (D), Glutamic acid (E); and (3) Asparagine (N), Glutamine (Q).

For example, it is understood that alterations in a nucleotide sequence, which reflect the degeneracy of the genetic code, or which result in the production of a chemically equivalent amino acid at a given site, are contemplated. Thus, a codon for the amino acid alanine, a hydrophobic amino acid, may be substituted by a codon encoding another less hydrophobic residue, such as glycine, or a more hydrophobic residue, such as valine, leucine, or isoleucine. Similarly, changes which result in substitution of one negatively charged residue for another, such as aspartic acid for glutamic acid, or one positively charged residue for another, such as lysine for arginine, can also be expected to produce a biologically equivalent product.

Nucleotide changes which result in alteration of the amino-terminal and carboxy-terminal portions of the encoded polypeptide molecule would also not generally be expected to alter the activity of the polypeptide. In some cases, it may in fact be desirable to make mutations in the sequence in order to study the effect of alteration on the biological activity of the polypeptide. Each of the proposed modifications is well within the routine skill in the art.

When the nucleic acid is prepared or altered synthetically, one of ordinary skill in the art can take into account the known codon preferences for the intended host where the nucleic acid is to be expressed. For example, although nucleic acid sequences of the present invention may be expressed in both monocotyledonous and dicotyledonous plant species, sequences can be modified to account for the specific codon preferences and GC-content preferences of monocotyledonous plants or dicotyledonous plants, as these preferences have been shown to differ.⁵¹

Cloning Techniques

For purposes of promoting an understanding of the principles of the invention, reference will now be made to particular embodiments of the invention and specific language will be used to describe the same. The materials, methods and examples are illustrative only and not limiting. Unless mentioned otherwise, the techniques employed or contemplated herein are standard methodologies well known to one of ordinary skill in the art. Specific terms, while employed below and defined at the end of this section, are used in a descriptive sense only and not for purposes of limitation. The practice of the present invention will employ, unless otherwise indicated, conventional techniques of botany, microbiology, tissue culture, molecular biology, chemistry, biochemistry and recombinant DNA technology, which are within the skill of the art.⁵²⁻⁵⁹

A suitable polynucleotide for use in accordance with the invention may be obtained by cloning techniques using cDNA or genomic libraries, DNA, or cDNA from bacteria which are available commercially or which may be constructed using standard methods known to persons of ordinary skill in the art. Suitable nucleotide sequences may be isolated from DNA libraries obtained from a wide variety of species by means of nucleic acid hybridization or amplification methods, such as polymerase chain reaction (PCR) procedures, using as probes or primers nucleotide sequences selected in accordance with the invention.

Furthermore, nucleic acid sequences may be constructed or amplified using chemical synthesis. The product of amplification is termed an amplicon. Moreover, if the particular nucleic acid sequence is of a length that makes chemical synthesis of the entire length impractical, the sequence may be broken up into smaller segments that may be synthesized and ligated together to form the entire desired sequence by methods known in the art. Alternatively, individual components or DNA fragments may be amplified by PCR and adjacent fragments can be amplified together using fusion-PCR,⁶⁰ overlap-PCR⁶¹ or chemical synthesis⁶²⁻⁶⁵ or using a vendor (e.g. GE life technologies, GENEART, Gen9, GenScript) by methods known in the art.

A suitable polynucleotide for use in accordance with the invention may be constructed by recombinant DNA technology, for example, by cutting or splicing nucleic acids using restriction enzymes and mixing with a cleaved (cut with a restriction enzyme) vector with the cleaved insert (DNA of the invention) and ligated using DNA ligase. Alternatively amplification techniques, such as PCR, can be used, where restriction sites are incorporated in the primers that otherwise match the nucleotide sequences (especially at the 3′ ends) selected in accordance with the invention. The desired amplified recombinant molecule is cut or spliced using restriction enzymes and mixed with a cleaved vector and ligated using DNA ligase. In another method, after amplification of the desired recombinant molecule, DNA linker sequences are ligated to the 5′ and 3′ ends of the desired nucleotide insert with ligase, the DNA insert is cleaved with a restriction enzyme that specifically recognizes sequences present in the linker sequences and the desired vector. The cleaved vector is mixed with the cleaved insert, and the two fragments are ligated using DNA ligase. In yet another method, the desired recombinant molecule is amplified with primers that have recombination sites (e.g. Gateway) incorporated in the primers, that otherwise match the nucleotide sequences selected in accordance with the invention. The desired amplified recombinant molecule is mixed with a vector containing the recombination site and recombinase, the two molecules are ligated together by recombination.

The recombinant expression cassette or DNA construct includes a promoter that directs transcription in a plant cell, operably linked to the polynucleotide encoding a CDO or SAD. In various aspects of the invention described herein, a variety of different types of promoters are described and used. As used herein, a polynucleotide is “operably linked” to a promoter or other nucleotide sequence when it is placed into a functional relationship with the promoter or other nucleotide sequence. The functional relationship between a promoter and a desired polynucleotide insert typically involves the polynucleotide and the promoter sequences being contiguous such that transcription of the polynucleotide sequence will be facilitated. Two nucleic acid sequences are further said to be operably linked if the nature of the linkage between the two sequences does not (1) result in the introduction of a frame-shift mutation; (2) interfere with the ability of the promoter region sequence to direct the transcription of the desired nucleotide sequence, or (3) interfere with the ability of the desired nucleotide sequence to be transcribed by the promoter sequence region. Typically, the promoter element is generally upstream (i.e., at the 5′ end) of the nucleic acid insert coding sequence.

While a promoter sequence can be ligated to a coding sequence prior to insertion into a vector, in other embodiments, a vector is selected that includes a promoter operable in the host cell into which the vector is to be inserted. In addition, certain preferred vectors have a region that codes a ribosome binding site positioned between the promoter and the site at which the DNA sequence is inserted so as to be operatively associated with the DNA sequence of the invention to produce the desired polypeptide, i.e., the DNA sequence of the invention in-frame.

Suitable Linkers

Peptide linkers are known to those skilled in the art to connect protein domains or peptides. In general linkers that contain the amino acids glycine and serine are useful linkers.^{66, 67} Other suitable linkers that can be used in the invention include, but are not limited to, those described by Kuusinen et. al. (1995),⁶⁸ Robinson and Sauer (1998),⁶⁹ Armstrong & Gouaux (2000),⁷⁰ Arai et. al. (2001),⁷¹ Wriggers et. al. (2005),⁷² and Reddy et. al. (2013).⁷³

Suitable Promoters

A wide variety of promoters are known to those of ordinary skill in the art as are other regulatory elements that can be used alone or in combination with promoters. A wide variety of promoters that direct transcription in plants cells can be used in connection with the present invention. For purposes of describing the present invention, promoters are divided into two types, namely, constitutive promoters and non-constitutive promoters. Constitutive promoters are classified as providing for a range of constitutive expression. Thus, some are weak constitutive promoters, and others are strong constitutive promoters. Non-constitutive promoters include tissue-preferred promoters, tissue-specific promoters, cell-type specific promoters, and inducible-promoters.

Inducible-promoters that respond to various forms of environmental stresses, or other stimuli, including, for example, mechanical shock, heat, cold, salt, flooding, drought, salt, anoxia, pathogens, such as bacteria, fungi, and viruses, and nutritional deprivation, including deprivation during times of flowering and/or fruiting, and other forms of plant stress. For example, the promoter selected in alternate forms of the invention, can be a promoter which is induced by one or more, but not limiting to one of the following, abiotic stresses such as wounding, cold, dessication, ultraviolet-B,⁷⁴ heat shock⁷⁵ or other heat stress, drought stress or water stress. The promoter may further be one induced by biotic stresses including pathogen stress, such as stress induced by a virus⁷⁶ or fungi,^{77, 78} stresses induced as part of the plant defense pathway⁷⁹ or by other environmental signals, such as light,⁸⁰ carbon dioxide^{81, 82}, hormones or other signaling molecules such as auxin, hydrogen peroxide and salicylic acid,^{83, 84} sugars and gibberellin⁸⁵ or abscisic acid and ethylene.⁸⁶

In other embodiments of the invention, tissue-specific promoters are used. Tissue-specific expression patterns as controlled by tissue- or stage-specific promoters that include, but is not limited to, fiber-specific, green tissue-specific, root-specific, stem-specific, and flower-specific. Examples of the utilization of tissue-specific expression includes, but is not limited to, the expression in leaves of the desired peptide for the protection of plants against foliar pathogens, the expression in roots of the desired peptide for the protection of plants against root pathogens, and the expression in roots or seedlings of the desired peptide for the protection of seedlings against soil-borne pathogens. In many cases, however, protection against more than one type of pathogen may be sought, and expression in multiple tissues will be desirable.

Of particular interest in certain embodiments of the present invention seed-specific promoters are used. Examples of the utilization of seed-specific promoters for expression includes, but is not limited to, napin,⁸⁷ sunflower seed-specific promoter,⁸⁸ AtFAD2,⁸⁹ phaseolin,⁹⁰ beta-conglycinin,⁹¹ zein,⁹² and rice glutelin.⁹³

Although some promoters from dicotyledons have been shown to be operational in monocotyledons and vice versa, ideally dicotyledonous promoters are selected for expression in dicotyledons, and monocotyledonous promoters are selected for expression in monocotyledons. There are also promoters that control expression of genes in green tissue or for genes involved in photosynthesis from both monocotyledons and dicotyledons such as the phosphenol carboxylase gene from maize.⁹⁴ There are suitable promoters for root specific expression.^{95, 96} A selected promoter can be an endogenous promoter, i.e. a promoter native to the species and or cell type being transformed. Alternatively, the promoter can be a foreign promoter, which promotes transcription of a length of DNA of viral, microbes, bacterial or eukaryotic origin, invertebrates, vertebrates including those from plants and plant viruses. For example, in certain preferred embodiments, the promoter may be of viral origin, including a cauliflower mosaic virus promoter (CaMV), such as CaMV 35S, a figwort mosaic virus promoter (FMV), or the coat protein promoter of tobacco mosaic virus (TMV). The promoter may further be, for example, a promoter for the small subunit of ribulose-1,3-biphosphate carboxylase. Promoters of bacterial origin include the octopine synthase promoter, the nopaline synthase promoter and other promoters derived from native Ti plasmids could also be utilized⁹⁷.

The promoters may further be selected such that they require activation by other elements known to those of ordinary skill in the art, so that production of the protein encoded by the nucleic acid sequence insert may be regulated as desired. In one embodiment of the invention, a DNA construct comprising a non-constitutive promoter operably linked to a polynucleotide encoding the desired polypeptide of the invention is used to make a transformed plant that selectively increases the level of the desired polypeptide of the invention in response to a signal. The term “signal” is used to refer to a condition, stress or stimulus that results in or causes a non-constitutive promoter to direct expression of a coding sequence operably linked to it. To make such a plant in accordance with the invention, a DNA construct is provided that includes a non-constitutive promoter operably linked to a polynucleotide encoding the desired polypeptide of the invention. The construct is incorporated into a plant genome to provide a transformed plant that expresses the polynucleotide in response to a signal.

In alternate embodiments of the invention, the selected promoter is a tissue-preferred promoter, a tissue-specific promoter, a cell-type-specific promoter, an inducible promoter or other type of non-constitutive promoter. It is readily apparent that such a DNA construct causes a plant transformed thereby to selectively express the gene for the desired polypeptide of the invention. Therefore under specific conditions or in certain tissue- or cell-types the desired polypeptide will be expressed. The result of this expression in the plant depends upon the activity of the promoter and in some cases the conditions of the cell or cells in which it is expressed.

It is understood that the non-constitutive promoter does not continuously produce the transcript or RNA of the invention. But in this embodiment the selected promoter for inclusion of the invention advantageously induces or increases transcription of the gene for the desired polypeptide of the invention in response to a signal, such as an environmental cue or other stress signal including biotic and/or abiotic stresses or other conditions.

In another embodiment of the invention, a DNA construct comprising a plant promoter operably linked to polynucleotides that encode the desired polypeptide of the invention is used to make a transformed plant that selectively increases the transcript or RNA of the desired polypeptide of the invention in the same cells, tissues, and under the environmental conditions that express a plant glutamate decarboxylase. It is understood to those of ordinary skill in the art that the regulatory sequences that comprise a plant promoter driven by RNA polymerase II reside in the region approximately 2900 to 1200 basepairs up-stream (5′) of the translation initiation site or start codon (ATG). For example, the full-length promoter for the nodule-enhanced PEP carboxylase from alfalfa is 1277 basepairs prior to the start codon,⁹⁸ the full-length promoter for cytokinin oxidase from orchid is 2189 basepairs prior to the start codon,⁹⁹ the full-length promoter for ACC oxidase from peach is 2919 basepairs prior to the start codon,¹⁰⁰ full-length promoter for cytokinin oxidase from orchid is 2189 basepairs prior to the start codon, full-length promoter for glutathione peroxidase1 from Citrus sinensis is 1600 basepairs prior to the start codon,¹⁰¹ and the full-length promoter for glucuronosyltransferase from cotton is 1647 basepairs prior to the start codon.¹⁰² Most full-length promoters are 1700 basepairs prior to the start codon. The accepted convention is to describe this region (promoter) as −1700 to −1, where the numbers designate the number of basepairs prior to the “A” in the start codon. However, regions less than 1700 basepairs prior to the start codon may be used. A promoter for these purposes normally means the following regions upstream (5′) to the start codon between −150 to −1 basepairs, preferably at least between −500 to −1 basepairs, preferably at least between −1000 to −1 basepairs, more preferably at least between −1500 to −1 basepairs, and most preferably at −2000 to −1 basepairs.

Plastid Transit Peptides

A wide variety of plastid transit peptides are known to those of ordinary skill in the art that can be used connection with the present invention. Suitable transit peptides which can be used to target any CDO polypeptide and/or SAD polypeptide to a plastid include, but are not limited, to those described herein and in U.S. Pat. Nos. 8,779,237, 8,674,180, 8,420,888, and 8,138,393 and in Lee et al.¹⁸⁴ and von Heijne et al.¹⁸⁵ Cloning a nucleic acid sequence encoding a transit peptide upstream and in-frame of a nucleic acid sequence encoding a polypeptide (for example, a CDO and/or SAD lacking its own transit peptide), involves standard molecular techniques that are well-known in the art.

Suitable Vectors

A wide variety of vectors may be employed to transform a plant, plant cell or other cells with a construct made or selected in accordance with the invention, including high- or low-copy number plasmids, phage vectors and cosmids. Such vectors, as well as other vectors, are well known in the art. Representative T-DNA vector systems^{97, 103} and numerous expression cassettes and vectors and in vitro culture methods for plant cell or tissue transformation and regeneration of plants are known and available.¹⁰⁴ The vectors can be chosen such that operably linked promoter and polynucleotides that encode the desired polypeptide of the invention are incorporated into the genome of the plant. Although the preferred embodiment of the invention is expression in plants or plant cells, other embodiments may include expression in prokaryotic or eukaryotic photosynthetic organisms, microbes, invertebrates or vertebrates.

It is known by those of ordinary skill in the art that there exist numerous expression systems available for expression of a nucleic acid encoding a protein of the present invention. There are many commercially available recombinant vectors to transform a host plant or plant cell. Standard molecular and cloning techniques^{56, 59, 105} are available to make a recombinant expression cassette that expresses the polynucleotide that encodes the desired polypeptide of the invention. No attempt to describe in detail the various methods known for the expression of proteins in prokaryotes or eukaryotes will be made. In brief, the expression of isolated nucleic acids encoding a protein of the present invention will typically be achieved by operably linking, for example, the DNA or cDNA to a promoter, followed by incorporation into an expression vector. The vectors can be suitable for replication and integration in either prokaryotes or eukaryotes. Typical expression vectors contain transcription and translation terminators, initiation sequences, and promoters useful for regulation of the expression of the DNA encoding a protein of the present invention. To obtain high-level expression of a cloned gene, it is desirable to construct expression vectors that contain, at the minimum, a strong promoter, such as ubiquitin, to direct transcription, a ribosome-binding site for translational initiation, and a transcription/translation terminator.

One of ordinary skill in the art recognizes that modifications could be made to a protein of the present invention without diminishing its biological activity. Some modifications may be made to facilitate the cloning, expression, targeting or to direct the location of the polypeptide in the host, or for the purification or detection of the polypeptide by the addition of a “tag” as a fusion protein. Such modifications are well known to those of skill in the art and include, for example, a Met added at the amino terminus to provide an initiation site, additional amino acids (tags) placed on either terminus to create a tag, additional nucleic acids to insert a restriction site or a termination.

In addition to the selection of a suitable promoter, the DNA constructs requires an appropriate transcriptional terminator to be attached downstream of the desired gene of the invention for proper expression in plants. Several such terminators are available and known to persons of ordinary skill in the art. These include, but are not limited to, the tml from CaMV and E9 from rbcS. Another example of a terminator sequence is the polyadenlyation sequence from the bovine growth hormone gene. A wide variety of available terminators known to function in plants can be used in the context of this invention. Vectors may also have other control sequence features that increase their suitability. These include an origin of replication, enhancer sequences, ribosome binding sites, RNA splice sites, polyadenylation sites, selectable markers and RNA stability signal. Origin of replication is a gene sequence that controls replication of the vector in the host cell. Enhancer sequences cooperate with the promoter to increase expression of the polynucleotide insert coding sequence. Enhancers can stimulate promoter activity in host cell. An example of specific polyadenylation sequence in higher eukaryotes is ATTTA. Examples of plant polyadenylation signal sequences are AATAAA or AATAAT. RNA splice sites are sequences that ensure accurate splicing of the transcript. Selectable markers usually confer resistance to an antibiotic, herbicide or chemical or provide color change, which aid the identification of transformed organisms. The vectors also include a RNA stability signal, which are 3′-regulatory sequence elements that increase the stability of the transcribed RNA.^{106, 107}

In addition, polynucleotides that encode a CDO or SAD can be placed in the appropriate plant expression vector used to transform plant cells. The polypeptide can then be isolated from plant callus or the transformed cells can be used to regenerate transgenic plants. Such transgenic plants can be harvested, and the appropriate tissues can be subjected to large-scale protein extraction and purification techniques.

The vectors may include another polynucleotide insert that encodes a peptide or polypeptide used as a “tag” to aid in purification or detection of the desired protein. The additional polynucleotide is positioned in the vector such that upon cloning and expression of the desired polynucleotide a fusion, or chimeric, protein is obtained. The tag may be incorporated at the amino or carboxy terminus. If the vector does not contain a tag, persons with ordinary skill in the art know that the extra nucleotides necessary to encode a tag can be added with the ligation of linkers, adaptors, or spacers or by PCR using designed primers. After expression of the peptide the tag can be used for purification using affinity chromatography, and if desired, the tag can be cleaved with an appropriate enzyme. The tag can also be maintained, not cleaved, and used to detect the accumulation of the desired polypeptide in the protein extracts from the host using western blot analysis. In another embodiment, a vector includes the polynucleotide for the tag that is fused in-frame to the polynucleotide that encodes a functional CDO or SAD to form a fusion protein. The tags that may be used include, but are not limited to, Arg-tag, calmodulin-binding peptide, cellulose-binding domain, DsbA, c-myc-tag, glutathione S-transferase, FLAG-tag, HAT-tag, His-tag, maltose-binding protein, NusA, S-tag, SBP-tag, Strep-tag, and thioredoxin (Trx-Tag). These are available from a variety of manufacturers Clontech Laboratories, Takara Bio Company GE Healthcare, Invitrogen, Novagen, Promega and QIAGEN.

The vector may include another polynucleotide that encodes a signal polypeptide or signal sequence to direct the desired polypeptide in the host cell, so that the polypeptide accumulates in a specific cellular compartment, subcellular compartment, or membrane. The specific cellular compartments include plastids or chloroplasts. A signal polypeptide or signal sequence is usually at the amino terminus and normally absent from the mature protein due to protease that removes the signal peptide when the polypeptide reaches its final destination. Signal sequences can be a primary sequence located at the N-terminus¹⁰⁸⁻¹¹¹, C-terminus^{112, 113} or internal¹¹⁴⁻¹¹⁶ or tertiary structure¹¹⁶. If a signal polypeptide or signal sequence to direct the polypeptide does not exist on the vector, it is expected that those of ordinary skill in the art can incorporate the extra nucleotides necessary to encode a signal polypeptide or signal sequence by the ligation of the appropriate nucleotides or by PCR. Those of ordinary skill in the art can identify the nucleotide sequence of a signal polypeptide or signal sequence using computational tools. There are numerous computational tools available for the identification of targeting sequences or signal sequence. These include, but are not limited to, TargetP^{117, 118}, iPSORT¹¹⁹, SignalP¹²⁰, PrediSi¹²¹, ELSpred¹²², HSLpred¹²³ and PSLpred¹²⁴, MultiLoc¹²⁵, SherLoc¹²⁶, ChloroP¹²⁷, MITOPROT¹²⁸, Predotar¹²⁹ and 3D-PSSM¹³⁰. Additional methods and protocols are discussed in the literature¹²⁵.

Transformation of Host Cells

Transformation of a plant can be accomplished in a wide variety of ways within the scope of a person of ordinary skill in the art. In one embodiment, a DNA construct is incorporated into a plant by (i) transforming a cell, tissue or organ from a host plant with the DNA construct; (ii) selecting a transformed cell, cell callus, somatic embryo, or seed which contains the DNA construct; (iii) regenerating a whole plant from the selected transformed cell, cell callus, somatic embryo, or seed; and (iv) selecting a regenerated whole plant that expresses the polynucleotide. Many methods of transforming a plant, plant tissue or plant cell for the construction of a transformed cell are suitable. Once transformed, these cells can be used to regenerate transgenic plants.¹³¹

Those of ordinary skill in the art can use different plant gene transfer techniques found in references for, but not limited to, the electroporation,¹³²⁻¹³⁶ microinjection,^{137, 138} lipofection¹³⁹ liposome or spheroplast fusions,¹⁴⁰⁻¹⁴² Agrobacterium,¹⁴³ direct gene transfer,¹⁴⁴ T-DNA mediated transformation of monocots,¹⁴⁵ T-DNA mediated transformation of dicots,^{146, 147} microprojectile bombardment or ballistic particle acceleration,¹⁴⁸⁻¹⁵¹ chemical transfection including CaCl₂ precipitation, polyvinyl alcohol, or poly-L-ornithine,¹⁵² silicon carbide whisker methods,^{153, 154} laser methods,^{155, 156} sonication methods,¹⁵⁷⁻¹⁵⁹ polyethylene glycol methods,¹⁶⁰ vacuum infiltration,¹⁶¹ and transbacter.¹⁶²

In one embodiment of the invention, a transformed host cell may be cultured to produce a transformed plant. In this regard, a transformed plant can be made, for example, by transforming a cell, tissue or organ from a host plant with an inventive DNA construct; selecting a transformed cell, cell callus, somatic embryo, or seed which contains the DNA construct; regenerating a whole plant from the selected transformed cell, cell callus, somatic embryo, or seed; and selecting a regenerated whole plant that expresses the polynucleotide.

A wide variety of host cells may be used in the invention, including prokaryotic and eukaryotic host cells. These cells or organisms may include microbes, invertebrate, vertebrates or photosynthetic organisms. Preferred host cells are eukaryotic, preferably plant cells, such as those derived from monocotyledons, such as duckweed, corn, rye grass, Bermuda grass, Blue grass, Fescue, or dicotyledons, including lettuce, cereals such as wheat, rapeseed, radishes and cabbage, green peppers, potatoes and tomatoes, and legumes such as soybeans and bush beans.

Suitable Plants

The methods described above may be applied to transform a wide variety of plants, including decorative or recreational plants or crops, but are particularly useful for treating commercial and ornamental crops. Examples of plants that may be transformed in the present invention include, but are not limited to, Acacia, alfalfa, aneth, apple, apricot, artichoke, arugula, asparagus, avocado, banana, barley, beans, beech, beet, Bermuda grass, bent grass, blackberry, blueberry, Blue grass, broccoli, Brussels sprouts, cabbage, canola, cantaloupe, carinata, carrot, cassava, cauliflower, celery, cherry, chicory, cilantro, citrus, clementines, coffee, corn, cotton, cucumber, duckweed, Douglas fir, eggplant, endive, escarole, eucalyptus, fennel, fescue, figs, forest trees, garlic, gourd, grape, grapefruit, honey dew, jicama, kiwifruit, lettuce, leeks, lemon, lime, Loblolly pine, maize, mango, melon, mushroom, nectarine, nut, oat, okra, onion, orange, an ornamental plant, papaya, parsley, pea, peach, peanut, pear, pepper, persimmon, pine, pineapple, plantain, plum, pomegranate, poplar, potato, pumpkin, quince, radiata pine, radicchio, radish, rapeseed, raspberry, rice, rye, rye grass, scallion, sorghum, Southern pine, soybean, spinach, squash, strawberry, sugar beet, sugarcane, sunflower, sweet potato, sweetgum, switchgrass, tangerine, tea, tobacco, tomato, turf, turnip, a vine, watermelon, wheat, yams, and zucchini Other suitable hosts include bacteria, fungi, algae and other photosynthetic organisms, and animals including vertebrate and invertebrates.

Once transformed, the plant may be treated with other “active agents” either prior to or during the exposure of the plant to stress to further decrease the effects of plant stress. “Active agent,” as used herein, refers to an agent that has a beneficial effect on the plant or increases production of amino acid production by the plant. For example, the agent may have a beneficial effect on the plant with respect to nutrition, and the resistance against, or reduction of, the effects of plant stress. Some of these agents may be precursors of end products for reaction catalyzed by CDO or SAD. These compounds could promote growth, development, biomass and yield, and change in metabolism. In addition to the twenty amino acids that are involved in protein synthesis specifically sulfur containing amino acids, Met and Cys, sulfur containing compounds such as sulfite, sulfate, taurine, hypotaurine, homotaurine, or N-acetyl thiozolidin 4 carboxylic acid (aminofol), or other non-protein amino acids, such as GABA, citrulline and ornithine, or other nitrogen containing compounds such as polyamines may also be used to activate CDO or SAD. Depending on the type of gene construct or recombinant expression cassette, other metabolites and nutrients may be used to activate CDO or SAD. These include, but are not limited to, sugars, carbohydrates, lipids, oligopeptides, mono- (glucose, arabinose, fructose, xylose, and ribose) di- (sucrose and trehalose) and polysaccharides, carboxylic acids (succinate, malate and fumarate) and nutrients such as phosphate, molybdate, or iron.

Accordingly, the active agent may include a wide variety of fertilizers, pesticides and herbicides known to those of ordinary skill in the art¹⁶³. Other greening agents fall within the definition of “active agent” as well, including minerals such as calcium, magnesium and iron. The pesticides protect the plant from pests or disease and may be either chemical or biological and include fungicides, bactericides, insecticides and anti-viral agents as known to those of ordinary skill in the art.

Expression in Prokaryotes

The use of prokaryotes as hosts includes strains of E. coli. However, other microbial strains including, but not limited to, Bacillus¹⁶⁴ and Salmonella may also be used. Commonly used prokaryotic control sequences include promoters for transcription initiation, optionally with an operator, along with ribosome binding site sequences. Commonly used prokaryotic promoters include the beta lactamase,¹⁶⁵ lactose,¹⁶⁵ and tryptophan¹⁶⁶ promoters. The vectors usually contain selectable markers to identify transfected or transformed cells. Some commonly used selectable markers include the genes for resistance to ampicillin, tetracycline, or chloramphenicol. The vectors are typically a plasmid or phage. Bacterial cells are transfected or transformed with the plasmid vector DNA. Phage DNA can be infected with phage vector particles or transfected with naked phage DNA. The plasmid and phage DNA for the vectors are commercially available from numerous vendors known to those of ordinary skill in the art.

Expression in Non-Plant Eukaryotes

The present invention can be expressed in a variety of eukaryotic expression systems such as yeast, insect cell lines, and mammalian cells which are known to those of ordinary skill in the art. For each host system there are suitable vectors that are commercially available (e.g., Invitrogen, Stratagene, GE Healthcare Life Sciences). The vectors usually have expression control sequences, such as promoters, an origin of replication, enhancer sequences, termination sequences, ribosome binding sites, RNA splice sites, polyadenylation sites, transcriptional terminator sequences, and selectable markers. Synthesis of heterologous proteins in yeast is well known to those of ordinary skill in the art.^{167, 168} The most widely used yeasts are Saccharomyces cerevisiae and Pichia pastoris. Insect cell lines that include, but are not limited to, mosquito larvae, silkworm, armyworm, moth, and Drosophila cell lines can be used to express proteins of the present invention using baculovirus-derived vectors. Mammalian cell systems often will be in the form of monolayers of cells although mammalian cell suspensions may also be used. A number of suitable host cell lines capable of expressing intact proteins have been developed in the art, and include the HEK293, BHK21, and CHO cell lines.

A protein of the present invention, once expressed in any of the non-plant eukaryotic systems can be isolated from the organism by lysing the cells and applying standard protein isolation techniques to the lysates or the pellets. The monitoring of the purification process can be accomplished by using western blot techniques or radioimmunoassay of other standard immunoassay techniques.

### DEFINITIONS

The term “polynucleotide” refers to a natural or synthetic linear and sequential array of nucleotides and/or nucleosides, including deoxyribonucleic acid, ribonucleic acid, and derivatives thereof. It includes chromosomal DNA, self-replicating plasmids, infectious polymers of DNA or RNA and DNA or RNA that performs a primarily structural role. Unless otherwise indicated, nucleic acids or polynucleotide are written left to right in 5′ to 3′ orientation, Nucleotides are referred to by their commonly accepted single-letter codes. Numeric ranges are inclusive of the numbers defining the range.

The terms “amplified” and “amplification” refer to the construction of multiple copies of a nucleic acid sequence or multiple copies complementary to the nucleic acid sequence using at least one of the nucleic acid sequences as a template. Amplification can be achieved by chemical synthesis using any of the following methods, such as solid-phase phosphoramidate technology or the polymerase chain reaction (PCR). Other amplification systems include the ligase chain reaction system, nucleic acid sequence based amplification, Q-Beta Replicase systems, transcription-based amplification system, and strand displacement amplification. The product of amplification is termed an amplicon.

As used herein “promoter” includes reference to a region of DNA upstream from the start of transcription and involved in recognition and binding of RNA polymerase, either I, II or III, and other proteins to initiate transcription. Promoters include necessary nucleic acid sequences near the start site of transcription, such as, in the case of a polymerase II type promoter, a TATA element. A promoter also optionally includes distal enhancer or repressor elements, which can be located as far as several thousand base pairs from the start site of transcription.

The term “plant promoter” refers to a promoter capable of initiating transcription in plant cells.

The term “foreign promoter” refers to a promoter, other than the native, or natural, promoter, which promotes transcription of a length of DNA of viral, bacterial or eukaryotic origin, including those from microbes, plants, plant viruses, invertebrates or vertebrates.

The term “microbe” refers to any microorganism (including both eukaryotic and prokaryotic microorganisms), such as fungi, yeast, bacteria, actinomycetes, algae and protozoa, as well as other unicellular structures.

The term “plant” includes whole plants, and plant organs, and progeny of same. Plant organs comprise, e.g., shoot vegetative organs/structures (e.g. leaves, stems and tubers), roots, flowers and floral organs/structures (e.g. bracts, sepals, petals, stamens, carpels, anthers and ovules), seed (including embryo, endosperm, and seed coat) and fruit (the mature ovary), plant tissue (e.g. vascular tissue, ground tissue, and the like) and cells (e.g. guard cells, egg cells, trichomes and the like). The class of plants that can be used in the method of the invention is generally as broad as the class of higher and lower plants amenable to transformation techniques, including angiosperms (monocotyledonous and dicotyledonous plants), gymnosperms, ferns, and multicellular algae. It includes plants of a variety of ploidy levels, including aneuploid, polyploid, diploid, haploid and hemizygous.

The term “peptide linker” refers to a peptide is used to join two peptides together. The peptide linker is derived from polynucleotide sequence inserted or cloned in-frame to join two peptides together as a fusion peptide.

The term “constitutive” refers to a promoter that is active under most environmental and developmental conditions, such as, for example, but not limited to, the CaMV 35S promoter and the nopaline synthase terminator.

The term “tissue-preferred promoter” refers to a promoter that is under developmental control or a promoter that preferentially initiates transcription in certain tissues.

The term “tissue-specific promoter” refers to a promoter that initiates transcription only in certain tissues.

The term “cell-type specific promoter” refers to a promoter that primarily initiates transcription only in certain cell types in one or more organs.

The term “inducible promoter” refers to a promoter that is under environmental control.

The term “plastid” refers to the class of plant cell organelles that includes amyloplasts, chloroplasts, chromoplasts, elaioplasts, eoplasts, etioplasts, leucoplasts, and proplastids.

The term “transit peptide” means a polypeptide that directs the transport of a nuclear encoded protein to a plastid. Typically, the transit peptide sequence is located at the N-terminus of a polypeptide, such as CDO or SAD.

The terms “encoding” and “coding” refer to the process by which a polynucleotide, through the mechanisms of transcription and translation, provides the information to a cell from which a series of amino acids can be assembled into a specific amino acid sequence to produce a functional polypeptide, such as, for example, an active enzyme or ligand binding protein.

The terms “polypeptide,” “peptide,” “protein” and “gene product” are used interchangeably herein to refer to a polymer of amino acid residues. The terms apply to amino acid polymers in which one or more amino acid residue is an artificial chemical analogue of a corresponding naturally occurring amino acid, as well as to naturally occurring amino acid polymers. Amino acids may be referred to by their commonly known three-letter or one-letter symbols. Amino acid sequences are written left to right in amino to carboxy orientation, respectively. Numeric ranges are inclusive of the numbers defining the range.

The terms “residue,” “amino acid residue,” and “amino acid” are used interchangeably herein to refer to an amino acid that is incorporated into a protein, polypeptide, or peptide. The amino acid may be a naturally occurring amino acid and may encompass known analogs of natural amino acids that can function in a similar manner as the naturally occurring amino acids.

The terms “cysteine dioxygenase” and “CDO” refer to the protein (EC 1.13.11.20) that catalyzes the following reactions:

cysteine+oxygen=3-sulfinoalanine

NOTE: 3-sulfinoalanine is another name for cysteine sulfinic acid, cysteine sulfinate, 3-sulphino-L-alanine, 3-sulfino-alanine, 3-sulfino-L-alanine, L-cysteine sulfinic acid, L-cysteine sulfinic acid, cysteine hydrogen sulfite ester or alanine 3-sulfinic acid

The terms “sulfinoalanine decarboxylase” and “SAD” refer to the protein (EC 4.1.1.29) that catalyzes the following reaction:

3-sulfinoalanine=hypotaurine+CO₂

NOTE: SAD is another name for cysteine-sulfinate decarboxylase, L-cysteine sulfinic acid decarboxylase, cysteine-sulfinate decarboxylase, CADCase/CSADCase, CSAD, cysteic decarboxylase, cysteine sulfinic acid decarboxylase, cysteine sulfinate decarboxylase, sulfoalanine decarboxylase, sulphinoalanine decarboxylase, and 3-sulfino-L-alanine carboxy-lyase.

NOTE: the SAD reaction is also catalyzed by GAD (4.1.1.15) (glutamic acid decarboxylase or glutamate decarboxylase).

Other names for hypotaurine are 2-aminoethane sulfinate, 2-aminoethylsulfinic acid, and 2-aminoethanesulfinic acid.

Other names for taurine are 2-aminoethane sulfonic acid, aminoethanesulfonate, L-taurine, taurine ethyl ester, and taurine ketoisocaproic acid 2-aminoethane sulfinate.

The term “functional” with reference to CDO or SAD refers to peptides, proteins or enzymes that catalyze the CDO or SAD reactions, respectively.

The term “plant-derived material” any part of the plant or a plant extract that is used directly or in part alone or as an additive or supplement. The material can be obtained through any one of the following processes that include, but is not limited to, crushed, pressed, pulverized milled, powdered, pounded, minced or extracted.

The term “recombinant” includes reference to a cell or vector that has been modified by the introduction of a heterologous nucleic acid. Recombinant cells express genes that are not normally found in that cell or express native genes that are otherwise abnormally expressed, under-expressed, or not expressed at all as a result of deliberate human intervention, or expression of the native gene may have reduced or eliminated as a result of deliberate human intervention.

The term “recombinant expression cassette” refers to a nucleic acid construct, generated recombinantly or synthetically, with a series of specified nucleic acid elements, which permit transcription of a particular nucleic acid in a target cell. The recombinant expression cassette can be incorporated into a plasmid, chromosome, plastid DNA, virus, or nucleic acid fragment. Typically, the recombinant expression cassette portion of an expression vector includes, among other sequences, a nucleic acid to be transcribed, and a promoter.

The term “transgenic plant” includes reference to a plant, which comprises within its genome a heterologous polynucleotide. Generally, the heterologous polynucleotide is integrated within the genome such that the polynucleotide is passed on to successive generations. The heterologous polynucleotide may be integrated into the genome alone or as part of a recombinant expression cassette. “Transgenic” is also used to include any cell, cell line, callus, tissue, plant part or plant, the genotype of which has been altered by the presence of heterologous nucleic acid including those transgenic plants altered or created by sexual crosses or asexual propagation from the initial transgenic plant. The term “transgenic” does not encompass the alteration of the genome by conventional plant breeding methods or by naturally occurring events such as random cross-fertilization, non-recombinant viral infection, non-recombinant bacterial transformation, non-recombinant transposition, or spontaneous mutation.

The term “vector” includes reference to a nucleic acid used in transfection or transformation of a host cell and into which can be inserted a polynucleotide.

The term “selectively hybridizes” includes reference to hybridization, under stringent hybridization conditions, of a nucleic acid sequence to a specified nucleic acid target sequence to a detectably greater degree (e.g., at least 2-fold over background) than its hybridization to non-target nucleic acid sequences and to the substantial exclusion of non-target nucleic acids. Selectively hybridizing sequences typically have about at least 40% sequence identity, preferably 60-90% sequence identity, and most preferably 100% sequence identity (i.e., complementary) with each other.

The terms “stringent conditions” and “stringent hybridization conditions” include reference to conditions under which a probe will hybridize to its target sequence, to a detectably greater degree than other sequences (e.g., at least 2-fold over background). Stringent conditions are sequence-dependent and will be different in different circumstances. By controlling the stringency of the hybridization and/or washing conditions, target sequences can be identified which can be up to 100% complementary to the probe (homologous probing). Alternatively, stringency conditions can be adjusted to allow some mismatching in sequences so that lower degrees of similarity are detected (heterologous probing). Optimally, the probe is approximately 500 nucleotides in length, but can vary greatly in length from less than 500 nucleotides to equal to the entire length of the target sequence.

Typically, stringent conditions will be those in which the salt concentration is less than about 1.5 M Na ion, typically about 0.01 to 1.0 M Na ion concentration (or other salts) at pH 7.0 to 8.3 and the temperature is at least about 30° C. for short probes (e.g., 10 to 50 nucleotides) and at least about 60° C. for long probes (e.g., greater than 50 nucleotides). Stringent conditions may also be achieved with the addition of destabilizing agents such as formamide or Denhardt's. Low stringency conditions include hybridization with a buffer solution of 30 to 35% formamide, 1 M NaCl, 1% SDS (sodium dodecyl sulfate) at 37° C., and a wash in 1× to 2×SSC (20×SSC=3.0 M NaCl/0.3 M trisodium citrate) at 50 to 55° C. Moderate stringency conditions include hybridization in 40 to 45% formamide, 1 M NaCl, 1% SDS at 37° C., and a wash in 0.5× to 1×SSC at 55 to 60° C. High stringency conditions include hybridization in 50% formamide, 1 M NaCl, 1% SDS at 37° C., and a wash in 0.1×SSC at 60 to 65° C. Specificity is typically the function of post-hybridization washes, the critical factors being the ionic strength and temperature of the final wash solution. For DNA-DNA hybrids, the Tₘ can be approximated¹⁶⁹, where the Tₘ=81.5° C.+16.6 (log M)+0.41 (% GC)−0.61 (% form)−500/L; where M is the molarity of monovalent cations, % GC is the percentage of guanosine and cytosine nucleotides in the DNA, % form is the percentage of formamide in the hybridization solution, and L is the length of the hybrid in base pairs. Tₘ is the temperature (under defined ionic strength and pH) at which 50% of a complementary target sequence hybridizes to a perfectly matched probe. Tₘ is reduced by about 1° C. for each 1% of mismatching; thus, Tₘ, hybridization and/or wash conditions can be adjusted to hybridize to sequences of the desired identity. For example, if sequences with ≧90% identity are sought, the Tₘ can be decreased 10° C. Generally, stringent conditions are selected to be about 5° C. lower than the thermal melting point (Tₘ) for the specific sequence and its complement at a defined ionic strength and pH. However, severely stringent conditions can utilize a hybridization and/or wash at 1, 2, 3 or 4° C. lower than the thermal melting point (Tₘ); moderately stringent conditions can utilize a hybridization and/or wash at 6, 7, 8, 9 or 10° C. lower than the thermal melting point (Tₘ); low stringency conditions can utilize a hybridization and/or wash at 11, 12, 13, 14, 15 or 20° C. lower than the thermal melting point (Tₘ). Using the equation, hybridization and wash compositions, and desired Tₘ, those of ordinary skill in the art will understand that variations in the stringency of hybridization and/or wash solutions are inherently described. An extensive guide to the hybridization of nucleic acids is found in the scientific literature.^{105, 170} Unless otherwise stated, in the present application high stringency is defined as hybridization in 4×SSC, 5×Denhardt's (5 g Ficoll, 5 g polyvinypyrrolidone, 5 g bovine serum albumin in 500 ml of water), 0.1 mg/ml boiled salmon sperm DNA, and 25 mM Na phosphate at 65° C., and a wash in 0.1×SSC, 0.1% SDS at 65° C.

The following terms are used to describe the sequence relationships between two or more nucleic acids or polynucleotides or polypeptides: “reference sequence,” “comparison window,” “sequence identity,” “percentage of sequence identity,” and “substantial identity.”

The term “reference sequence” is a defined sequence used as a basis for sequence comparison. A reference sequence may be a subset or the entirety of a specified sequence; for example, as a segment of a full-length cDNA or gene sequence, or the complete cDNA or gene sequence.

The term “comparison window” includes reference to a contiguous and specified segment of a polynucleotide sequence, where the polynucleotide sequence may be compared to a reference sequence and the portion of the polynucleotide sequence in the comparison window may comprise additions or deletions (i.e., gaps) when it is compared to the reference sequence for optimal alignment. The comparison window is usually at least 20 contiguous nucleotides in length, and optionally can be 30, 40, 50, 100 or longer. Those of ordinary skill in the art understand that the inclusion of gaps in a polynucleotide sequence alignment introduces a gap penalty, and it is subtracted from the number of matches.

Methods of alignment of nucleotide and amino acid sequences for comparison are well known to those of ordinary skill in the art. The local homology algorithm, BESTFIT,¹⁷¹ can perform an optimal alignment of sequences for comparison using a homology alignment algorithm called GAP¹⁷², search for similarity using Tfasta and Fasta¹⁷³, by computerized implementations of these algorithms widely available on-line or from various vendors (Intelligenetics, Genetics Computer Group). CLUSTAL allows for the alignment of multiple sequences¹⁷⁴⁻¹⁷⁶ and program PileUp can be used for optimal global alignment of multiple sequences.¹⁷⁷ The BLAST family of programs can be used for nucleotide or protein database similarity searches. BLASTN searches a nucleotide database using a nucleotide query. BLASTP searches a protein database using a protein query. BLASTX searches a protein database using a translated nucleotide query that is derived from a six-frame translation of the nucleotide query sequence (both strands). TBLASTN searches a translated nucleotide database using a protein query that is derived by reverse-translation. TBLASTX search a translated nucleotide database using a translated nucleotide query.

GAP¹⁷² maximizes the number of matches and minimizes the number of gaps in an alignment of two complete sequences. GAP considers all possible alignments and gap positions and creates the alignment with the largest number of matched bases and the fewest gaps. It also calculates a gap penalty and a gap extension penalty in units of matched bases. Default gap creation penalty values and gap extension penalty values in Version 10 of the Wisconsin Genetics Software Package are 8 and 2, respectively. The gap creation and gap extension penalties can be expressed as an integer selected from the group of integers consisting of from 0 to 100. GAP displays four figures of merit for alignments: Quality, Ratio, Identity, and Similarity. The Quality is the metric maximized in order to align the sequences. Ratio is the quality divided by the number of bases in the shorter segment. Percent Identity is the percent of the symbols that actually match. Percent Similarity is the percent of the symbols that are similar. Symbols that are across from gaps are ignored. A similarity is scored when the scoring matrix value for a pair of symbols is greater than or equal to 0.50, the similarity threshold. The scoring matrix used in Version 10 of the Wisconsin Genetics Software Package is BLOSUM62.¹⁷⁸

Unless otherwise stated, sequence identity or similarity values refer to the value obtained using the BLAST 2.0 suite of programs using default parameters.¹⁷⁹ As those of ordinary skill in the art understand that BLAST searches assume that proteins can be modeled as random sequences and that proteins comprise regions of nonrandom sequences, short repeats, or enriched for one or more amino acid residues, called low-complexity regions. These low-complexity regions may be aligned between unrelated proteins even though other regions of the protein are entirely dissimilar. Those of ordinary skill in the art can use low-complexity filter programs to reduce number of low-complexity regions that are aligned in a search. These filter programs include, but are not limited to, the SEG180, 181 and XNU.¹⁸²

The terms “sequence identity” and “identity” are used in the context of two nucleic acid or polypeptide sequences and include reference to the residues in the two sequences, which are the same when aligned for maximum correspondence over a specified comparison window. When the percentage of sequence identity is used in reference to proteins it is recognized that residue positions which are not identical often differ by conservative amino acid substitutions, where amino acid residues are substituted for other amino acid residues with similar chemical properties (e.g., charge or hydrophobicity) and therefore do not change the functional properties of the molecule. Where sequences differ in conserved substitutions, the percent sequence identity may be adjusted upwards to correct for the conserved nature of the substitution. Sequences, which differ by such conservative substitutions, are said to have “sequence similarity” or “similarity.” Scoring for a conservative substitution allows for a partial rather than a full mismatch,¹⁸³ thereby increasing the percentage sequence similarity.

The term “percentage of sequence identity” means the value determined by comparing two optimally aligned sequences over a comparison window, wherein the portion of the polynucleotide sequence in the comparison window may comprise gaps (additions or deletions) when compared to the reference sequence for optimal alignment. The percentage is calculated by determining the number of positions at which the identical nucleic acid base or amino acid residue occurs in both sequences to yield the number of matched positions, dividing the number of matched positions by the total number of positions in the window of comparison and multiplying the result by 100 to yield the percentage of sequence identity.

The term “substantial identity” of polynucleotide sequences means that a polynucleotide comprises a sequence that has between 50-100% sequence identity, preferably at least 50% sequence identity, preferably at least 60% sequence identity, preferably at least 70%, more preferably at least 80%, more preferably at least 90%, and most preferably at least 95%, compared to a reference sequence using one of the alignment programs described using standard parameters. One of ordinary skill in the art will recognize that these values can be appropriately adjusted to determine corresponding identity of proteins encoded by two nucleotide sequences by taking into account codon degeneracy, amino acid similarity, reading frame positioning and the like. Substantial identity of amino acid sequences for these purposes normally means sequence identity of between 50-100%. Another indication that nucleotide sequences are substantially identical is if two molecules hybridize to each low stringency conditions, moderate stringency conditions or high stringency conditions. Yet another indication that two nucleic acid sequences are substantially identical is if the two polypeptides immunologically cross-react with the same antibody in a western blot, immunoblot or ELISA assay.

The terms “substantial identity” in the context of a peptide indicates that a peptide comprises a sequence with between 55-100% sequence identity to a reference sequence preferably at least 55% sequence identity, preferably 60% preferably 70%, more preferably 80%, most preferably at least 90% or 95% sequence identity to the reference sequence over a specified comparison window. Preferably, optimal alignment is conducted using the homology alignment algorithm.¹⁷² Thus, a peptide is substantially identical to a second peptide, for example, where the two peptides differ only by a conserved substitution. Another indication that amino acid sequences are substantially identical is if two polypeptides immunologically cross-react with the same antibody in a western blot, immunoblot or ELISA assay. In addition, a peptide can be substantially identical to a second peptide when they differ by a non-conservative change if the epitope that the antibody recognizes is substantially identical.

All patents, patent applications, and references cited in this disclosure are expressly incorporated herein by reference. The above disclosure generally describes the present invention. A more complete understanding can be obtained by reference to the following specific examples, which are provided for purposes of illustration only and are not intended to limit the scope of the invention.

### Example 1

### Development of a Transgenic Plant that Constitutively Expresses (35S Promoter) CDO with a Plastid Transit Peptide

Step 1: Use chemical synthesis to make a DNA construct that contains a constitutive promoter, 35S, fused with the nucleotide sequence for a plastid transit peptide (SEQ ID NO:9), CDO gene (SEQ ID NO:1 or 2) and a NOS terminator. Clone the DNA construct into a binary vector, such as pCambia1300, pCambia2300 or pCambia3200. The nucleotide sequence for the plastid transit peptide (SEQ ID NO:9) encodes the peptide SEQ ID NO:10.

The CDO genes are as follows:
a. Derived from SEQ ID NO:1 optimized for expression in Arabidopsis, a dicot, or corn a, monocot, and encodes a CDO peptide (SEQ ID NO:3) from bovine;
b. Derived from SEQ ID NO:2 optimized for expression in Arabidopsis, a dicot, or corn, a monocot, and encodes a CDO peptide (SEQ ID NO:4) from Danio rerio.

Step 2: Transform Agrobacterium tumefaciens: Transform the DNA construct into Agrobacterium tumefaciens, select for antibiotic resistance and confirm the presence of the DNA construct.

Step 3: Transform plant (Arabidopsis, soybean, corn, wheat, sugar beet, rice, camelina or canola), select for antibiotic resistance, select for transgenic plants. Confirm the presence of the DNA construct in the transgenic plants.

### Example 2

### Development of a Transgenic Plant that Constitutively Expresses (35S Promoter) CDO with a Plastid Transit Peptide and (35S Promoter) SAD with a Plastid Transit Peptide (CDO and SAD are in Tandem with Independent Promoters)

Step 1: Use chemical synthesis to make a DNA construct that contains a constitutive promoter, 35S, fused with the nucleotide sequence for a plastid transit peptide (SEQ ID NO: 9), CDO gene (SEQ ID NO: 1 or 2) and a NOS terminator. Clone the DNA construct into a binary vector, such as pCambia1300, pCambia2300 or pCambia3200. The nucleotide sequence for the plastid transit peptide (SEQ ID NO: 9) encodes the peptide SEQ ID NO: 10.

The CDO genes are as follows:
a. Derived from SEQ ID NO:1 optimized for expression in Arabidopsis, a dicot, or corn, a monocot, and encodes a CDO peptide (SEQ ID NO:3) from bovine;
b. Derived from SEQ ID NO:2 optimized for expression in Arabidopsis, a dicot, or corn, a monocot, and encodes a CDO peptide (SEQ ID NO:4) from Danio rerio.

Step 2: Use chemical synthesis to make a DNA construct that contains a constitutive promoter, 35S, fused with the nucleotide sequence for a plastid transit peptide (SEQ ID NO: 29), SAD gene (SEQ ID NO: 5 or 6) and a NOS terminator. The nucleotide sequence for the plastid transit peptide (SEQ ID NO: 9) encodes the peptide SEQ ID NO: 10. Clone the SAD DNA construct into a binary vector that contains the CDO DNA construct (Step1).

The SAD genes are as follows:
a. Derived from SEQ ID NO:5 optimized for expression in Arabidopsis, a dicot, or corn, a monocot, and encodes a SAD peptide (SEQ ID NO:7) from horse;
b. Derived from SEQ ID NO:6 optimized for expression in Arabidopsis, a dicot, or corn, a monocot, and encodes a CDO peptide (SEQ ID NO:8) from Danio rerio.

Step 3: Transform Agrobacterium tumefaciens: Transform the DNA construct into Agrobacterium tumefaciens, select for antibiotic resistance and confirm the presence of the DNA construct.

Step 4: Transform plant (Arabidopsis, soybean, corn, wheat, sugar beet, rice, camelina or canola), select for antibiotic resistance, select for transgenic plants. Confirm the presence of the DNA construct in the transgenic plants

### Example 3

### Development of a Transgenic Plant that Constitutively Expresses (35S Promoter) CDO Fused (without a Linker) to SAD with a Transit Peptide Using Chemical Synthesis

Step 1: Use chemical synthesis to make a DNA construct that contains a constitutive promoter, 35S, fused with nucleotide sequence for a plastid transit peptide (SEQ ID NO: 29), CDO gene (SEQ ID NO: 1 or 2), and SAD gene (SEQ ID NO: 5 or 6) all in-frame and a NOS terminator. Clone the DNA construct into a binary vector, such as pCambia1300, pCambia2300 or pCambia3200. The nucleotide sequence for the plastid transit peptide (SEQ ID NO: 9) encodes the peptide SEQ ID NO: 10.

The CDO genes are as follows:
a. Derived from SEQ ID NO:1 optimized for expression in Arabidopsis, a dicot, or corn, a monocot, and encodes a CDO peptide (SEQ ID NO:3) from bovine;
b. Derived from SEQ ID NO:2 optimized for expression in Arabidopsis, a dicot, or corn, a monocot, and encodes a CDO peptide (SEQ ID NO:4) from Danio rerio.

The SAD genes are as follows:
a. Derived from SEQ ID NO:5 optimized for expression in Arabidopsis, a dicot, or corn, a monocot, and encodes a SAD peptide (SEQ ID NO:7) from horse;
b. Derived from SEQ ID NO:6 optimized for expression in Arabidopsis, a dicot, or corn, a monocot, and encodes a CDO peptide (SEQ ID NO:8) from Danio rerio.

Step 2: Transform Agrobacterium tumefaciens: Transform the DNA construct into Agrobacterium tumefaciens, select for antibiotic resistance and confirm the presence of the DNA construct.

Step 3: Transform plant (Arabidopsis, soybean, corn, wheat, sugar beet, rice, camelina or canola), select for antibiotic resistance, select for transgenic plants. Confirm the presence of the DNA construct in the transgenic plants.

### Example 4

### Development of a Transgenic Plant that Constitutively Expresses (35S Promoter) CDO Fused to SAD with a Linker with a Plastid Transit Peptide

Step 1: Use chemical synthesis to make a DNA construct that contains a constitutive promoter, 35S, fused with nucleotide sequence for a plastid transit peptide (SEQ ID NO: 29), CDO gene (SEQ ID NO: 1 or 2), a linker (SEQ ID NO:11), SAD gene (SEQ ID NO: 5 or 6) all in-frame and a NOS terminator. Clone the plastid transit-CDO-linker-SAD DNA construct into a binary vector, such as pCambia1300, pCambia2300 or pCambia3200. The nucleotide sequence for the plastid transit peptide (SEQ ID NO: 9) encodes the peptide SEQ ID NO: 10.

The CDO genes are as follows:
a. Derived from SEQ ID NO:1 optimized for expression in Arabidopsis, a dicot, or corn a, monocot, and encodes a CDO peptide (SEQ ID NO:3) from bovine;
b. Derived from SEQ ID NO:2 optimized for expression in Arabidopsis, a dicot, or corn, a monocot, and encodes a CDO peptide (SEQ ID NO:4) from Danio rerio.

The SAD genes are as follows:
a. Derived from SEQ ID NO:5 optimized for expression in Arabidopsis, a dicot, or corn, a monocot, and encodes a SAD peptide (SEQ ID NO:7) from horse;
b. Derived from SEQ ID NO:6 optimized for expression in Arabidopsis, a dicot, or corn, a monocot, and encodes a CDO peptide (SEQ ID NO:8) from Danio rerio.

Step 2: Transform Agrobacterium tumefaciens: Transform the DNA construct into Agrobacterium tumefaciens, select for antibiotic resistance and confirm the presence of the DNA construct.

Step 3: Transform plant (Arabidopsis, soybean, corn, wheat, sugar beet, rice, camelina or canola), select for antibiotic resistance, select for transgenic plants. Confirm the presence of the DNA construct in the transgenic plants.

### Example 5

### Increased Met Levels in Transgenic Plants with the Plastid Transit Peptide CDO Linked SAD Construct

Transgenic Arabidopsis plants that expressed CDO fused to SAD with a linker (CLS) either with a transit peptide (seedpro_plastCLS) or without a transit peptide (seedpro_CLS) using s seed-specific promoter were developed. Developed at the same time were empty vector control (EVC) plants, which were transgenic plants with the vector minus a gene insert. Amino acids were extracted from mature dry seeds (˜80-day-old). Table 1 shows the mean and median percent Met values (g/g dry weight) of the dry seed for each of the three groups. A Wilcoxon Rank-Sum Test showed statistically significantly higher (˜2 times) Met levels in the seedpro_plastCLS group compared to the EVC group, t(14)=2.2274, p<0.05. The Met levels of the seedpro_CLS were similar to those of the EVC group, t(7)=0.343, ns.

TABLE 3
Percent Met (g/g DW) in mature, dry seed

| | EVC | seedpro_PCLS | seedpro_CLS |
|---|---|---|---|
| n | 6 | 10 | 3 |
| Mean | 0.0034 | 0.0065 | 0.0038 |
| SD | 0.0020 | 0.0030 | 0.0013 |
| Median | 0.0029 | 0.0061 | 0.0042 |

The use of the terms “a” and “an” and “the” and similar referents in the context of describing the invention (especially in the context of the following claims) are to be construed to cover both the singular and the plural, unless otherwise indicated herein or clearly contradicted by context. The terms “comprising,” “having,” “including,” and “containing” are to be construed as open-ended terms (i.e., meaning “including, but not limited to,”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

Embodiments of this invention are described herein, including the best mode known to the inventors for carrying out the invention. Variations of those embodiments may become apparent to those of ordinary skill in the art upon reading the foregoing description. The inventors expect skilled artisans to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context. Embodiments of this invention are described herein, including the best mode known to the inventors for carrying out the invention. Variations of those embodiments may become apparent to those of ordinary skill in the art upon reading the foregoing description. The inventors expect skilled artisans to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

### BIBLIOGRAPHY

1. Hesse, H. & Hoefgen, R. Molecular aspects of methionine biosynthesis. Trends Plant Sci. 8, 259-262 (2003).
2. Grabau, L. J., Blevins, D. G. & Minor, H. C. Stem infusions enhanced methionine content of soybean storage protein. Plant Physiol. 82, 1013-1018 (1986).
3. Tabe, L. M. & Droux, M. Limits to sulfur accumulation in transgenic lupin seeds expressing a foreign sulfur-rich protein. Plant Physiol. 128, 1137-1148 (2002).
4. Bagga, S. et al. Genetic engineering ruminal stable high methionine protein in the foliage of alfalfa. Plant Sci. 166, 273-283 (2004).
5. Hacham, Y., Avraham, T. & Amir, R. The N-terminal region of Arabidopsis cystathionine gamma-synthase plays an important regulatory role in methionine metabolism. Plant Physiol. 128, 454-462 (2002).
6. Hacham, Y., Schuster, G. & Amir, R. An in vivo internal deletion in the N-terminus region of Arabidopsis cystathionine gamma-synthase results in CGS expression that is insensitive to methionine. Plant J. 45, 955-967 (2006).
7. Hacham, Y., Matityahu, I., Schuster, G. & Amir, R. Overexpression of mutated forms of aspartate kinase and cystathionine gamma-synthase in tobacco leaves resulted in the high accumulation of methionine and threonine. Plant J. 54, 260-271 (2008).
8. Di, R. et al. Enhancement of the primary flavor compound methional in potato by increasing the level of soluble methionine. J. Agric. Food Chem. 51, 5695-5702 (2003).
9. Dancs, G., Kondrak, M. & Banfalvi, Z. The effects of enhanced methionine synthesis on amino acid and anthocyanin content of potato tubers. BMC Plant Biol 8, 65 (2008).
10. Avraham, T., Badani, H., Galili, S. & Amir, R. Enhanced levels of methionine and cysteine in transgenic alfalfa (Medicago sativa L.) plants over-expressing the Arabidopsis cystathionine gamma-synthase gene. Plant Biotechnol J 3, 71-79 (2005).
11. Bartlem, D. et al. Mutation in the threonine synthase gene results in an over-accumulation of soluble methionine in Arabidopsis. Plant Physiol. 123, 101-110 (2000).
12. Zeh, M. et al. Antisense inhibition of threonine synthase leads to high methionine content in transgenic potato plants. Plant Physiol. 127, 792-802 (2001).
13. Avraham, T. & Amir, R. The expression level of threonine synthase and cystathionine-gamma-synthase is influenced by the level of both threonine and methionine in Arabidopsis plants. Transgenic research 14, 299-311 (2005).
14. Nguyen, H. C., Hoefgen, R. & Hesse, H. Improving the nutritive value of rice seeds: elevation of cysteine and methionine contents in rice plants by ectopic expression of a bacterial serine acetyltransferase. J. Exp. Bot. 63, 5991-6001 (2012).
15. Wandelt, C. I. et al. Vicilin with carboxy-terminal KDEL is retained in the endoplasmic reticulum and accumulates to high levels in the leaves of transgenic plants. Plant J. 2, 181-192 (1992).
16. Ealing, P. M., Hancock, K. R. & White, D. W. Expression of the pea albumin 1 gene in transgenic white clover and tobacco. Transgenic research 3, 344-354 (1994).
17. Nordlee, J. A., Taylor, S. L., Townsend, J. A., Thomas, L. A. & Bush, R. K. Identification of a Brazil-nut allergen in transgenic soybeans. The New England journal of medicine 334, 688-692 (1996).
18. Cobbett, C. S. Phytochelatin biosynthesis and function in heavy-metal detoxification. Curr. Opin. Plant Biol. 3, 211-216 (2000).
19. Beinert, H. A tribute to sulfur. Eur. J. Biochem. 267, 5657-5664 (2000).
20. Leustek, T., Martin, M. N., Bick, J. A. & Davies, J. P. PATHWAYS AND REGULATION OF SULFUR METABOLISM REVEALED THROUGH MOLECULAR AND GENETIC STUDIES. Annu. Rev. Plant Physiol. Plant Mol. Biol. 51, 141-165 (2000).
21. Saito, K. Regulation of sulfate transport and synthesis of sulfur-containing amino acids. Curr. Opin. Plant Biol. 3, 188-195 (2000).
22. Giovanelli, J., Mudd, S. H. & Datko, A. H. in The Biochemistry of Plants, Vol. 5. (ed. B. J. Miflin) 453-505 (Academic Press, New York; 1980).
23. Schmidt, A. & Jager, K. OPEN QUESTIONS ABOUT SULFUR METABOLISM IN PLANTS. Annu. Rev. Plant Physiol. Plant Mol. Biol. 43, 325-349 (1992).
24. Wirtz, M., Droux, M. & Hell, R. O-acetylserine (thiol) lyase: an enigmatic enzyme of plant cysteine biosynthesis revisited in Arabidopsis thaliana. J. Exp. Bot. 55, 1785-1798 (2004).
25. Heeg, C. et al. Analysis of the Arabidopsis O-acetylserine(thiol)lyase gene family demonstrates compartment-specific differences in the regulation of cysteine synthesis. The Plant Cell 20, 168-185 (2008).
26. Romero, L. C. et al. Cysteine and Cysteine-Related Signaling Pathways in Arabidopsis thaliana. Molecular plant 7, 264-276 (2014).
27. Blaszczyk, A., Brodzik, R. & Sirko, A. Increased resistance to oxidative stress in transgenic tobacco plants overexpressing bacterial serine acetyltransferase. The Plant Journal 20, 237-243 (1999).
28. Harms, K., von Ballmoos, P., Brunold, C., Hofgen, R. & Hesse, H. Expression of a bacterial serine acetyltransferase in transgenic potato plants leads to increased levels of cysteine and glutathione. Plant J. 22, 335-343 (2000).
29. Wirtz, M. & Hell, R. Production of cysteine for bacterial and plant biotechnology: application of cysteine feedback-insensitive isoforms of serine acetyltransferase. Amino Acids 24, 195-203 (2003).
30. Hopkins, L. et al. O-acetylserine and the regulation of expression of genes encoding components for sulfate uptake and assimilation in potato. Plant Physiol. 138, 433-440 (2005).
31. Krueger, S. et al. Analysis of cytosolic and plastidic serine acetyltransferase mutants and subcellular metabolite distributions suggests interplay of the cellular compartments for cysteine biosynthesis in Arabidopsis. Plant Cell Environ. 32, 349-367 (2009).
32. Amir, R., Hacham, Y. & Galili, G. Cystathionine gamma-synthase and threonine synthase operate in concert to regulate carbon flow towards methionine in plants. Trends Plant Sci. 7, 153-156 (2002).
33. Hacham, Y., Avraham, T. & Amir, R. The N-terminal region of Arabidopsis cystathionine gamma synthase plays an important role in methionine metabolism. Plant Physiol. 128, 454-462 (2002).
34. Di, R. et al. Enhancement of the primary flavor compound methional in potato by increasing the level of soluble methionine. Journal of Agriculture and Food Chemistry 51, 5695-5702 (2003).
35. Avraham, T., Badani, H., Galili, S. & Amir, R. Enhanced levels of methionine and cysteine in transgenic alfalfa (Medicago sativa L.) plants overexpressing the Arabidopsis cystathionine c-synthase gene. Plant Biotechnology Journal 3, 71-80 (2005).
36. Fontecave, M., Atta, M. & Mulliez, E. S-adenosylmethionine: nothing goes to waste. Trends in biochemical sciences 29, 243-249 (2004).
37. McKean, T. A. & Yang, S. F. in Plant Hormones and Their Role in Plant Growth and Development. (ed. P. J. Davies) 97 (Kluwer Academic Publishers, Boston; 1988).
38. Kende, H. Ethylene Biosynthesis. Annu. Rev. Plant Physiol. Plant Mol. Biol. 44, 283-307 (1993).
39. Morgan, P. W. & Drew, M. C. Ethylene and plant responses to stress. Physiol. Plant 100, 620-630 (1997).
40. Bleecker, A. B. & Kende, H. Ethylene: a gaseous signal molecule in plants. Annual Review of Cell and Developmental Biology 16, 1-18 (2000).
41. Tiburcio, A. F., Campos, J. L., Figueras, X. & Besford, R. T. Recent advances in the understanding of polyamine functions during plant development. Plant Growth Regul. 12, 331-340 (1993).
42. Bouchereau, A., Aziz, A., Larher, F. & Martin-Tanguy, J. Polyamines and environmental challenges: recent development. Plant Sci. 140, 103-125 (1999).
43. Roje, S. S-Adenosyl-L-methionine: beyond the universal methyl group donor. Phytochemistry 67, 1686-1698 (2006).
44. Kusano, T., Berberich, T., Tateda, C. & Takahashi, Y. Polyamines: essential factors for growth and survival. Planta 228, 367-381 (2008).
45. Boerjan, W., Bauw, G., Van Montagu, M. & Inze, D. Distinct phenotypes generated by overexpression and suppression of S-adenosyl-L-methionine synthetase reveal developmental patterns of gene silencing in tobacco. Plant Cell 6, 1401-1414 (1994).
46. Morandini, P. Rethinking metabolic control. Plant Sci. 176, 441-451 (2009).
47. Stitt, M., Sulpice, R. & Keurentjes, J. Metabolic Networks: How to Identify Key Components in the Regulation of Metabolism and Growth. Plant Physiol. 152, 428-444 (2010).
48. Morandini, P. Control limits for accumulation of plant metabolites: brute force is no substitute for understanding. Plant Biotechnology Journal 11, 253-267 (2013).
49. Honjoh, K. I. et al. Enhancement of menadione stress tolerance in yeast by accumulation of hypotaurine and taurine: co-expression of cDNA clones, from Cyprinus carpio, for cysteine dioxygenase and cysteine sulfinate decarboxylase in Saccharomyces cerevisiae. Amino Acids 38, 1173-1183 (2010).
50. Turano, F. J., Turano, K. A., Carlson, P. S. & Kinnersley, A. M. (Plant Sensory Systems, LLC, US; 2012).
51. Murray, E. E., Lotzer, J. & Eberle, M. Codon usage in plant genes. Nucleic Acids Research 17, 477-498 (1989).
52. Langenheim, J. H. & Thimann, K. V. Botany: Plant Biology and its Relation to Human Affairs. (John Wiley & Sons Inc., New York; 1982).
53. Vasil, I. K. Cell Culture and Somatic Cell Genetics of Plants: Laboraory Procedures and Their Applications, Vol. 1. (Academic Press, Orlando; 1984).
54. Stanier, R., Ingrahm, J., Wheelis, M. & Painter, P. The Microbial World, Edn. 5. (Prentice-Hall, New Jersey; 1986).
55. Dhringra, O. D. & Sinclair, J. B. Basic plant pathology methods. (CRC Press, Boca Raton, Fla.; 1985).
56. Maniatis, T., Fritsch, E. F. & Sambrook, J. Molecular Cloning: A Laboratory Manual: DNA Cloning, Vol. I and II. (Cold Spring Harbor, New York; 1985).
57. Gait Oligonucleotide Synthesis-A Practical Approach. (IRL Press, Washington, D.C.; 1984).
58. Hames, D. D. & Higgins, S. J. Nucleic Acid Hybridization: A Practical Approach. (IRL Press, Washington D.C.; 1984).
59. Watson, J. D., Gilman, M., Witowski, J. & Zoller, M. Recombinant DNA. (Scientific American Books, New York; 1992).
60. Szewczyk, E. et al. Fusion PCR and gene targeting in Aspergillus nidulans. Nat. Protoc. 1, 3111-3121 (2006).
61. Ho, S. N., Hunt, H. D., Horton, R. M., Pullen, J. K. & Pease, L. R. Site-directed mutagenesis by overlap extension using the polymerase chain reaction. Gene 77, 51-59 (1989).
62. Stemmer, W. P., Crameri, A., Ha, K. D., Brennan, T. M. & Heyneker, H. L. Single-step assembly of a gene and entire plasmid from large numbers of oligodeoxyribonucleotides. Gene 164, 49-53 (1995).
63. Gao, X., Yo, P., Keith, A., Ragan, T. J. & Harris, T. K. Thermodynamically balanced inside-out (TBIO) PCR-based gene synthesis: a novel method of primer design for high-fidelity assembly of longer gene sequences. Nucleic Acids Res. 31, e143 (2003).
64. Kodumal, S. J. et al. Total synthesis of long DNA sequences: synthesis of a contiguous 32-kb polyketide synthase gene cluster. Proc. Natl. Acad. Sci. U.S.A 101, 15573-15578 (2004).
65. Xiong, A.-S. et al. Chemical gene synthesis: strategies, softwares, error corrections, and applications. FEMS Microbiology Reviews 32, 522-540 (2008).
66. Trinh, R., Gurbaxani, B., Morrison, S. L. & Seyfzadeh, M. Optimization of codon pair use within the (GGGGS)3 linker sequence results in enhanced protein expression. Molecular immunology 40, 717-722 (2004).
67. Chang, T. W. & Yu, L. (Google Patents, 1999).
68. Kuusinen, A., Arvola, M. & Keinanen, K. Molecular dissection of the agonist binding site of an AMPA receptor. EMBO (Eur. Mol. Biol. Organ.) J. 14, 6327-6332 (1995).
69. Robinson, C. R. & Sauer, R. T. Optimizing the stability of single-chain proteins by linker length and composition mutagenesis. Proc. Natl. Acad. Sci. U.S.A 95, 5929-5934 (1998).
70. Armstrong, N. & Gouaux, E. Mechanisms for activation and antagonism of an AMPA-sensitive glutamate receptor: crystal structures of the GluR2 ligand binding core. Neuron 28, 165-181 (2000).
71. Arai, R., Ueda, H., Kitayama, A., Kamiya, N. & Nagamune, T. Design of the linkers which effectively separate domains of a bifunctional fusion protein. Protein Eng. 14, 529-532 (2001).
72. Wriggers, W., Chakravarty, S. & Jennings, P. A. Control of protein functional dynamics by peptide linkers. Biopolymers 80, 736-746 (2005).
73. Reddy Chichili, V. P., Kumar, V. & Sivaraman, J. Linkers in the structural biology of protein-protein interactions. Protein Sci. 22, 153-167 (2013).
74. van Der Krol, A. R. et al. Developmental and wound-, cold-, desiccation-, ultraviolet-B-stress-induced modulations in the expression of the petunia zinc finger transcription factor gene ZPT2-2. Plant Physiol. 121, 1153-1162 (1999).
75. Shinmyo, A. et al. Metabolic engineering of cultured tobacco cells. Biotechnol. Bioeng. 58, 329-332 (1998).
76. Sohal, A. K., Pallas, J. A. & Jenkins, G. I. The promoter of a Brassica napus lipid transfer protein gene is active in a range of tissues and stimulated by light and viral infection in transgenic Arabidopsis. Plant Mol. Biol. 41, 75-87 (1999).
77. Eulgem, T., Rushton, P. J., Schmelzer, E., Hahlbrock, K. & Somssich, I. E. Early nuclear events in plant defence signalling: rapid gene activation by WRKY transcription factors. EMBO (Eur. Mol. Biol. Organ.) J. 18, 4689-4699 (1999).
78. Cormack, R. S. et al. Leucine zipper-containing WRKY proteins widen the spectrum of immediate early elicitor-induced WRKY transcription factors in parsley. Biochim. Biophys. Acta 1576, 92-100 (2002).
79. Lebel, E. et al. Functional analysis of regulatory sequences controlling PR-1 gene expression in Arabidopsis. Plant J. 16, 223-233 (1998).
80. Ngai, N., Tsai, F. Y. & Coruzzi, G. Light-induced transcriptional repression of the pea AS1 gene: identification of cis-elements and transfactors. Plant J. 12, 1021-1034 (1997).
81. Kucho, K., Ohyama, K. & Fukuzawa, H. CO(2)-responsive transcriptional regulation of CAH1 encoding carbonic anhydrase is mediated by enhancer and silencer regions in Chlamydomonas reinhardtii. Plant Physiol. 121, 1329-1338 (1999).
82. Kucho, K., Yoshioka, S., Taniguchi, F., Ohyama, K. & Fukuzawa, H. Cis-acting elements and DNA-binding proteins involved in CO2-responsive transcriptional activation of Cah1 encoding a periplasmic carbonic anhydrase in Chlamydomonas reinhardtii. Plant Physiol. 133, 783-793 (2003).
83. Chen, W. & Singh, K. B. The auxin, hydrogen peroxide and salicylic acid induced expression of the Arabidopsis GST6 promoter is mediated in part by an ocs element. Plant J. 19, 667-677 (1999).
84. Chen, W., Chao, G. & Singh, K. B. The promoter of a H2O2-inducible, Arabidopsis glutathione S-transferase gene contains closely linked OBF- and OBP1-binding sites. Plant J. 10, 955-966 (1996).
85. Lu, C. A., Lim, E. K. & Yu, S. M. Sugar response sequence in the promoter of a rice alpha-amylase gene serves as a transcriptional enhancer. J. Biol. Chem. 273, 10120-10131 (1998).
86. Leubner-Metzger, G., Petruzzelli, L., Waldvogel, R., Vogeli-Lange, R. & Meins, F., Jr. Ethylene-responsive element binding protein (EREBP) expression and the transcriptional regulation of class I beta-1,3-glucanase during tobacco seed germination. Plant Mol. Biol. 38, 785-795 (1998).
87. Kridl, J. C. et al. Isolation and characterization of an expressed napin gene from Brassica rapa. Seed Science Research 1, 209-219 (1991).
88. Zavallo, D., Lopez Bilbao, M., Hopp, H. E. & Heinz, R. Isolation and functional characterization of two novel seed-specific promoters from sunflower (Helianthus annuus L.). Plant Cell Rep. 29, 239-248 (2010).
89. Kim, M. J. et al. Seed-specific expression of sesame microsomal oleic acid desaturase is controlled by combinatorial properties between negative cis-regulatory elements in the SeFAD2 promoter and enhancers in the 5′-UTR intron. Molecular genetics and genomics: MGG 276, 351-368 (2006).
90. Bustos, M. M. et al. Regulation of beta-glucuronidase expression in transgenic tobacco plants by an A/T-rich, cis-acting sequence found upstream of a French bean beta-phaseolin gene. Plant Cell 1, 839-853 (1989).
91. Fujiwara, T. & Beachy, R. N. Tissue-specific and temporal regulation of a beta-conglycinin gene: roles of the RY repeat and other cis-acting elements. Plant Mol. Biol. 24, 261-272 (1994).
92. Wienand, U., Langridge, P. & Feix, G. Isolation and characterization of a genomic sequence of maize coding for a zein gene. Molec. Gen. Genet. 182, 440-444 (1981).
93. Takaiwa, F., Oono, K., Wing, D. & Kato, A. Sequence of three members and expression of a new major subfamily of glutelin genes from rice. Plant Mol. Biol. 17, 875-885 (1991).
94. Hudspeth, R. L., Grula, J. W., Dai, Z., Edwards, G. E. & Ku, M. S. Expression of maize phosphoenolpyruvate carboxylase in transgenic tobacco: Effects on biochemistry and physiology. Plant Physiol. 98, 458-464 (1992).
95. de Framond, A. J. A metallothionein-like gene from maize (Zea mays). Cloning and characterization. FEBS Lett. 290, 103-106 (1991).
96. Hudspeth, R. L., Hobbs, S. L., Anderson, D. M., Rajasekaran, K. & Grula, J. W. Characterization and expression of metallothionein-like genes in cotton. Plant Mol. Biol. 31, 701-705 (1996).
97. Herrera-Estrella, L., Depicker, A., van Montagu, M. & Schell, J. Expression of chimaeric genes transferred into plant cells using a Ti-plasmid-derived vector. Nature 303, 209-213 (1983).
98. Pathirana, M. S. et al. Analyses of phosphoenolpyruvate carboxylase gene structure and expression in alfalfa nodules. Plant J. 12, 293-304 (1997).
99. Yang, S. H., Yu, H. & Goh, C. J. Isolation and characterization of the orchid cytokinin oxidase DSCKX1 promoter. J. Exp. Bot. 53, 1899-1907 (2002).
100. Moon, H. & Callahan, A. M. Developmental regulation of peach ACC oxidase promoter-GUS fusions in transgenic tomato fruits. J. Exp. Bot. 55, 1519-1528 (2004).
101. Avsian-Kretchmer, O., Gueta-Dahan, Y., Lev-Yadun, S., Gollop, R. & Ben-Hayyim, G. The salt-stress signal transduction pathway that activates the gpx1 promoter is mediated by intracellular H2O2, different from the pathway induced by extracellular H2O2. Plant Physiol. 135, 1685-1696 (2004).
102. Wu, A.-M., Lv, S.-Y. & Liu, J.-Y. Functional analysis of a cotton glucuronosyltransferase promoter in transgenic tobaccos. Cell Res. 17, 174-183 (2007).
103. An, G., Watson, B. D., Stachel, S., Gordon, M. P. & Nester, E. W. New cloning vehicles for transformation of higher plants. EMBO (Eur. Mol. Biol. Organ.) J. 4, 277-284 (1985).
104. Gruber, M. Y. & Cosby, W. L. in Methods in Plant Molecular Biology and Biotechnology. (eds. B. R. Glick & J. E. Thompson) 89-119 (CRC Press, Baco Raton, Fla.; 1993).
105. Ausubel, F. M. et al. Current Protocols in Molecular Biology. (Greene Publishing and Wiley-Interscience, New York; 1995).
106. Ohme-Takagi, M., Taylor, C. B., Newman, T. C. & Green, P. J. The effect of sequences with high AU content on mRNA stability in tobacco. Proc. Natl. Acad. Sci. U.S.A 90, 11811-11815 (1993).
107. Newman, T. C., Ohme-Takagi, M., Taylor, C. B. & Green, P. J. DST sequences, highly conserved among plant SAUR genes, target reporter transcripts for rapid decay in tobacco. Plant Cell 5, 701-714 (1993).
108. von Heijne, G. Mitochondrial targeting sequences may form amphiphilic helices. EMBO (Eur. Mol. Biol. Organ.) J. 5, 1335-1342 (1986).
109. Swinkels, B. W., Gould, S. J., Bodnar, A. G., Rachubinski, R. A. & Subramani, S. A novel, cleavable peroxisomal targeting signal at the amino-terminus of the rat 3-ketoacyl-CoA thiolase. EMBO (Eur. Mol. Biol. Organ.) J. 10, 3255-3262 (1991).
110. Rusch, S. L. & Kendall, D. A. Protein transport via amino-terminal targeting sequences: Common themes in diverse systems. Mol. Membr. Biol. 12, 295-307 (1995).
111. Soll, J. & Tien, R. Protein translocation into and across the chloroplastic envelope membranes. Plant Mol. Biol. 38, 191-207 (1998).
112. Gould, S. J., Keller, G. A. & Subramani, S. Identification of peroxisomal targeting signals located at the carboxy terminus of four peroxisomal proteins. J. Cell Biol. 107, 897-905 (1988).
113. Gould, S. J., Keller, G. A., Hosken, N., Wilkinson, J. & Subramani, S. A conserved tripeptide sorts proteins to peroxisomes. J. Cell Biol. 108, 1657-1664 (1989).
114. McCammon, M. T., McNew, J. A., Willy, P. J. & Goodman, J. M. An internal region of the peroxisomal membrane protein PMP47 is essential for sorting to peroxisomes. J. Cell Biol. 124, 915-925 (1994).
115. Cokol, M., Nair, R. & Rost, B. Finding nuclear localization signals. EMBO Rep. 1, 411-415 (2000).
116. Helenius, A. & Aebi, M. Intracellular functions of N-linked glycans. Science 291, 2364-2369 (2001).
117. Emanuelsson, O., Brunak, S., von Heijne, G. & Nielsen, H. Locating proteins in the cell using TargetP, SignalP and related tools. Nat. Protoc. 2, 953-971 (2007).
118. Emanuelsson, O., Nielsen, H., Brunak, S. & von Heijne, G. Predicting subcellular localization of proteins based on their N-terminal amino acid sequence. J. Mol. Biol. 300, 1005-1016 (2000).
119. Bannai, H., Tamada, Y., Maruyama, O., Nakai, K. & Miyano, S. Extensive feature detection of N-terminal protein sorting signals. Bioinformatics 18, 298-305 (2002).
120. Bendtsen, J. D., Nielsen, H., von Heijne, G. & Brunak, S. Improved prediction of signal peptides: SignalP 3.0. J. Mol. Biol. 340, 783-795 (2004).
121. Hiller, K., Grote, A., Scheer, M., Munch, R. & Jahn, D. PrediSi: prediction of signal peptides and their cleavage positions. Nucleic Acids Res. 32, W375-379 (2004).
122. Bhasin, M. & Raghava, G. P. ESLpred: SVM-based method for subcellular localization of eukaryotic proteins using dipeptide composition and PSI-BLAST. Nucleic Acids Res. 32, W414-419 (2004).
123. Garg, A., Bhasin, M. & Raghava, G. P. Support vector machine-based method for subcellular localization of human proteins using amino acid compositions, their order, and similarity search. J. Biol. Chem. 280, 14427-14432 (2005).
124. Bhasin, M., Garg, A. & Raghava, G. P. PSLpred: prediction of subcellular localization of bacterial proteins. Bioinformatics 21, 2522-2524 (2005).
125. Hoglund, A., Donnes, P., Blum, T., Adolph, H. W. & Kohlbacher, 0. MultiLoc: prediction of protein subcellular localization using N-terminal targeting sequences, sequence motifs and amino acid composition. Bioinformatics 22, 1158-1165 (2006).
126. Shatkay, H. et al. SherLoc: high-accuracy prediction of protein subcellular localization by integrating text and protein sequence data. Bioinformatics 23, 1410-1417 (2007).
127. Emanuelsson, O., Nielsen, H. & von Heijne, G. ChloroP, a neural network-based method for predicting chloroplast transit peptides and their cleavage sites. Protein Sci. 8, 978-984 (1999).
128. Claros, M. G. & Vincens, P. Computational method to predict mitochondrially imported proteins and their targeting sequences. Eur. J. Biochem. 241, 779-786 (1996).
129. Small, I., Peeters, N., Legeai, F. & Lurin, C. Predotar: A tool for rapidly screening proteomes for N-terminal targeting sequences. Proteomics 4, 1581-1590 (2004).
130. Kelley, L. A., MacCallum, R. M. & Sternberg, M. J. Enhanced genome annotation using structural profiles in the program 3D-PSSM. J. Mol. Biol. 299, 499-520 (2000).
131. Shahin, E. A. Totipotency of tomato protoplasts. Theon Appl Genet 69, 235-240 (1985).
132. Fromm, M., Taylor, L. P. & V., W. Expression of genes transferred into monocot and dicot plant cells by electroporation. Proc. Natl. Acad. Sci. U.S.A 82, 5824-5828 (1985).
133. Fromm, M. E., Taylor, L. P. & Walbot, V. Stable transformation of maize after gene transfer by electroporation. Nature 319, 791-793 (1986).
134. Riggs, C. D. & Bates, G. W. Stable transformation of tobacco by electroporation: evidence for plasmid concatenation. Proc. Natl. Acad. Sci. U.S.A 83, 5602-5606 (1986).
135. D'Halluin, K., Bonne, E., Bossut, M., De Beuckeleer, M. & Leemans, J. Transgenic maize plants by tissue electroporation. Plant Cell 4, 1495-1505 (1992).
136. Laursen, C. M., Krzyzek, R. A., Flick, C. E., Anderson, P. C. & Spencer, T. M. Production of fertile transgenic maize by electroporation of suspension culture cells Plant Mol. Biol. 24, 51-61 (1994).
137. Crossway, A. et al. Integration of foreign DNA following microinjection of tobacco mesophyll protoplasts. Mol. Gen. Genet. 202, 179-185 (1986).
138. Griesbach, R. J. Protoplast microinjection. Plant Mol. Biol. Report. 1, 32-37 (1983).
139. Sporlein, B. & Koop, H.-U. Lipofectin: direct gene transfer to higher plants using cationic liposomes. Theon Appl Genet 83, 1-5 (1991).
140. Ohgawara, T., Uchimiya, H. & Harada, H. Uptake of liposome-encapsulated plasmid DNA by plant protoplasts and molecular fate of foreign DNA Protoplasma 116, 145-148 (1983).
141. Deshayes, A., Herrera-Estrella, L. & Caboche, M. Liposome-mediated transformation of tobacco mesophyll protoplasts by an Escherichia coli plasmid. EMBO (Eur. Mol. Biol. Organ.) J. 4, 2731-2737 (1985).
142. Christou, P., Murphy, J. E. & Swain, W. F. Stable transformation of soybean by electroporation and root formation from transformed callus. Proc. Natl. Acad. Sci. U.S.A 84, 3962-3966 (1987).
143. Horsch, R. B. et al. A Simple and General Method for Transferring Genes into Plants. Science 227, 1229-1231 (1985).
144. Paszkowski, J. et al. Direct gene transfer to plants. The EMBO journal 3, 2717-2722 (1984).
145. Hooykaas-Van Slogteren, G. M., Hooykaas, P. J. & Schilperoort, R. A. Expression of Ti plasmid genes in monocotyledonous plants infected with Agrobacterium tumefaciens. Nature 311, 763-764 (1984).
146. Rogers, S. G., Horsch, R. B., and Fraley, R. T. 1986. Gene transfer in plants: Production of transformed plants using Ti-plasmid vectors. Gene transfer in plants: Production of transformed plants using Ti-plasmid vectors. Methods Enzymol. 118, 627-640 (1986).
147. Bevan, M. W. & Chilton, M.-D. T-DNA of the Agrobacterium Ti and Ri plasmids. Annu. Rev. Genet. 16, 357-384 (1982).
148. Klein, T. M. et al. Transfer of foreign genes into intact maize cells with high-velocity microprojectiles. Proc. Natl. Acad. Sci. U.S.A 85, 4305-4309 (1988).
149. Klein, T. M., Gradziel, T., Fromm, M. E. & Sanford, J. C. Factors influencing gene delivery into Zea mays cells by high-velocity microprojectiles. Biotechnology 6, 559-563 (1988).
150. McCabe, D. E., Swain, W. F., Martinell, B. J. & Christou, P. Stable transformation of soybean (Glycine max) by particle acceleration. Biotechnology 6, 923-926 (1988).
151. Sanford, J. C., Smith, F. D. & Rushell, J. A. in The Methods in Enzymology, Vol. 217. (ed. R. Wu) 483-509 (Academic Press, Orlando; 1993).
152. Freeman, J. P. et al. A Comparison of Methods for Plasmid Delivery into Plant Protoplasts. Plant Cell Physiol. 25, 1353-1365 (1984).
153. Frame, B. R. et al. Production of fertile transgenic maize plants by silicon carbide whisker-mediated transformation. Plant J. 6, 941-948 (1994).
154. Thompson, J. A., Drayton, P., Frame, B., Wang, K. & Dunwell, J. M. Maize transformation utilizing silicon carbide whiskers: a review. Euphytica 85, 75-80 (1995).
155. Guo, Y., Liang, H. & Berns, M. W. Laser-mediated gene transfer in rice. Physiol. Plant 93, 19-24 (1995).
156. Badr, Y. A., Kereim, M. A., Yehia, M. A., Fouad, O. O. & Bahieldin, A. Production of fertile transgenic wheat plants by laser micropuncture. Photochem. Photobiol. Sci. 4, 803-807 (2005).
157. Bao, S., Thrall, B. D. & Miller, D. L. Transfection of a reporter plasmid into cultured cells by sonoporation in vitro. Ultrasound in Medicine and Biology 23, 953-959 (1997).
158. Finer, K. R. & Finer, J. J. Use of Agrobacterium expressing green fluorescent protein to evaluate colonization of sonication-assisted Agrobacterium-mediated transformation-treated soybean cotyledons. Lett. Appl. Microbiol. 30, 406-410 (2000).
159. Amoah, B. K., Wu, H., Sparks, C. & Jones, H. D. Factors influencing Agrobacterium-mediated transient expression of uidA in wheat inflorescence tissue. J. Exp. Bot. 52, 1135-1142 (2001).
160. Krens, F. A., Molendijk, L., Wullems, G. J. & Schilperoort, R. A. In Vitro transformation of plant protoplasts with Ti-plasmid DNA. Nature 296, 72-74 (1982).
161. Bechtold, N. & Pelletier, G. In planta Agrobacterium-mediated transformation of adult Arabidopsis thaliana plants by vacuum infiltration. Methods Mol. Biol. 82, 259-266 (1998).
162. Broothaerts, W. et al. Gene transfer to plants by diverse species of bacteria. Nature 433, 629-633 (2005).
163. Kirk, R. E. & Othmer, D. F. Concise Encyclopedia of Chemical Technology, Edn. 4th. (John Wiley & Sons, 1993).
164. Mosbach, K., Birnbaum, S., Hardy, K., Davies, J. & Bülow, L. Formation of proinsulin by immobilized Bacillus subtilis. Nature 302, 543-545 (1983).
165. Chan, H. W. & Wells, R. D. Structural uniqueness of lactose operator. Nature 252, 205-209 (1974).
166. Goeddel, D. V. et al. Synthesis of human fibroblast interferon by E. coli Nucleic Acids Res. 8, 4057-4074 (1980).
167. Sherman, F., Fink, G. R. & Hick, J. B. Methods in Yeast Genetics. (Cold Spring Harbor Laboratory, New York; 1982).
168. Sherman, F. in Methods in Enzymology, Guide to Yeast Genetics and Molecular Biology, Vol. 194. (eds. C. Guthrie & G. R. Fink) 3-21 (Acad. Press, New York; 1991).
169. Meinkoth, J. & Wahl, G. Hybridization of nucleic acids immobilized on solid supports. Anal. Biochem. 138, 267-284 (1984).
170. Tijssen, P. in Laboratory Techniques in Biochemistry and Molecular Biology-Hybridization with Nucleic Acid Probes: Part I (Elsevier, New York; 1993).
171. Smith, T. F. & Waterman, M. S. Comparison of biosequences. Adv. Appl. Math. 2, 482-489 (1981).
172. Needleman, S. B. & Wunsch, C. D. A general method applicable to the search for similarities in the amino acid sequence of two proteins. J. Mol. Biol. 48, 443-453 (1970).
173. Pearson, W. R. & Lipman, D. J. Improved tools for biological sequence comparison. Proc. Natl. Acad. Sci. U.S.A 85, 2444-2448 (1988).
174. Higgins, D. G. & Sharp, P. M. Fast and sensitive multiple sequence alignments on a microcomputer. Comput. Appl. Biosci. 5, 151-153 (1989).
175. Higgins, D. G. & Sharp, P. M. CLUSTAL: a package for performing multiple sequence alignment on a microcomputer. Gene 73, 237-244 (1988).
176. Higgins, D. G., Bleasby, A. J. & Fuchs, R. CLUSTAL V: improved software for multiple sequence alignment. Comput. Appl. Biosci. 8, 189-191 (1992).
177. Feng, D. F. & Doolittle, R. F. Progressive sequence alignment as a prerequisite to correct phylogenetic trees. J. Mol. Evol. 25, 351-360 (1987).
178. Henikoff, S. & Henikoff, J. Amino acid substitution matrices from protein blocks Proc. Natl. Acad. Sci. U.S.A 89, 10915-10919 (1989).
179. Altschul, S. F. et al. Gapped BLAST and PSI-BLAST: a new generation of protein database search programs. Nucleic Acids Res. 25, 3389-3402 (1997).
180. Wootton, J. C. & Federhen, S. Statistics of local complexity in amino acid sequences and sequence databases. Comput. Chem. 17, 149-163 (1993).
181. Wootton, J. C. & Federhen, S. Analysis of compositionally biased regions in sequence databases. Methods Enzymol. 266, 554-571 (1996).
182. Claverie, J.-M. & States, D. J. Information enhancement methods for large scale sequence analysis. Comput. Chem. 17, 191-201 (1993).
183. Myers, E. W. & Miller, W. Optimal alignments in linear-space. Comput. Appl. Biol. Sci. 4, 11-17 (1988).
184. Lee, D. W. et al. Arabidopsis nuclear-encoded plastid transit peptides contain multiple sequence subgroups with distinctive chloroplast-targeting sequence motifs. Plant Cell 20, 1603-1622 (20208).
185. von Heinje, G. et al. CHLPEP—a database of chloroplast transit peptides. Plant Mol Biol Rept 9, 104-126 (1991).

What is claimed is:

1. A cell of a photosynthetic organism, an algae or a plant comprising either:
(a) two expression units, wherein
(i) a first expression unit comprises a first promoter operably linked to a first nucleic acid comprising (a) a first polynucleotide which encodes a plastid transit peptide operatively linked to (b) a second polynucleotide which encodes cysteine dioxygenase (CDO); and
(ii) a second expression unit comprises a second promoter operably linked to a second nucleic acid comprising (a) a third polynucleotide which encodes a plastid transit peptide operatively linked to (b) a fourth polynucleotide which encodes sulfinoalanine decarboxylase (SAD); or
(b) a single expression unit comprising a third promoter operably linked to a third nucleic acid comprising (a) a fifth polynucleotide which encodes a plastid transit peptide operatively linked to (b) a sixth polynucleotide which encodes either CDO or CDO fused to SAD,
wherein the cell has an increased level of Methionine compared to a cell not comprising the expression units.

2. The cell of claim 1, wherein the CDO comprises the amino acid sequence set forth in SEQ ID NO:3 or SEQ ID NO:4.

3. The cell of claim 1, wherein the SAD comprises the amino acid sequence set forth in SEQ ID NO:7 or SEQ ID NO:8.

4. The cell of claim 1, wherein the first polynucleotide or portion of the sixth polynucleotide encoding CDO comprises the nucleotide sequence set forth in SEQ ID NO:1 or SEQ ID NO:2.

5. The cell of claim 1, wherein the second polynucleotide or portion of the sixth polynucleotide encoding SAD comprises the nucleotide sequence set forth in SEQ ID NO:5 or SEQ ID NO:6.

6. The cell of claim 1, wherein at least one of the first, second, and third promoters is a constitutive promoter.

7. The cell of claim 1, wherein at least one of the first, second, and third promoters is a non-constitutive promoter.

8. The cell of claim 7, wherein the non-constitutive promoter is selected from the group consisting of a tissue-preferred promoter, a tissue-specific promoter, a seed-specific promoter, a cell type-specific promoter, or an inducible promoter.

9. The cell of claim 1, wherein the cell is in vitro.

10. The cell of claim 1, wherein the cell is in vivo.

11. The cell of claim 1, wherein the cell is a plant cell and the plant is selected from the group consisting of acacia, alfalfa, aneth, apple, apricot, artichoke, arugula, asparagus, avocado, banana, barley, beans, beech, beet, Bermuda grass, blackberry, blueberry, Blue grass, broccoli, Brussels sprouts, cabbage, canola, cantaloupe, carinata, carrot, cassava, cauliflower, celery, cherry, chicory, cilantro, citrus, clementine, coffee, corn, cotton, cucumber, duckweed, Douglas fir, eggplant, endive, escarole, eucalyptus, fennel, fescue, figs, forest trees, garlic, gourd, grape, grapefruit, honey dew, jicama, kiwifruit, lettuce, leeks, lemon, lime, Loblolly pine, maize, mango, melon, mushroom, nectarine, nut, oat, okra, onion, orange, an ornamental plant, papaya, parsley, pea, peach, peanut, pear, pepper, persimmon, pine, pineapple, plantain, plum, pomegranate, poplar, potato, pumpkin, quince, radiata pine, radicchio, radish, rapeseed, raspberry, rice, rye, rye grass, scallion, sorghum, Southern pine, soybean, spinach, squash, strawberry, sugar beet, sugarcane, sunflower, sweet potato, sweetgum, switchgrass, tangerine, tea, tobacco, tomato, turf, turnip, a vine, watermelon, wheat, yams, and zucchini.

12. A transgenic plant comprising the plant cell of claim 1, wherein the transgenic plant has an increased Methionine level compared to a plant not comprising the expression units.

13. A transgenic plant seed comprising the plant cell of claim 1, wherein the transgenic plant seed has an increased Methionine level compared to a plant seed not comprising the expression units.

14. A method for producing Methionine in a photosynthetic organism, an algae or a plant, comprising
incorporating into a cell of a photosynthetic organism, an algae or a plant either:
(a) two expression units, wherein
(i) a first expression unit comprises a first promoter operably linked to a first nucleic acid comprising (a) a first polynucleotide which encodes a plastid transit peptide operatively linked to (b) a second polynucleotide which encodes cysteine dioxygenase (CDO); and
(ii) a second expression unit comprises a second promoter operably linked to a second nucleic acid comprising (a) a third polynucleotide which encodes a plastid transit peptide operatively linked to (b) a fourth polynucleotide which encodes sulfinoalanine decarboxylase (SAD); or
(b) a single expression unit comprising a third promoter operably linked to a third nucleic acid comprising (a) a fifth polynucleotide which encodes a plastid transit peptide operatively linked to (b) a sixth polynucleotide which encodes either CDO or CDO fused to SAD and
growing the cell under suitable conditions to produce Methionine,
wherein the cell has an increased level of Methionine compared to a cell not comprising the expression units.

15. The method of claim 14, wherein the cell is a plant cell and the plant is selected from the group consisting of acacia, alfalfa, aneth, apple, apricot, artichoke, arugula, asparagus, avocado, banana, barley, beans, beech, beet, Bermuda grass, blackberry, blueberry, Blue grass, broccoli, Brussels sprouts, cabbage, canola, cantaloupe, carinata, carrot, cassava, cauliflower, celery, cherry, chicory, cilantro, citrus, clementine, coffee, corn, cotton, cucumber, duckweed, Douglas fir, eggplant, endive, escarole, eucalyptus, fennel, fescue, figs, forest trees, garlic, gourd, grape, grapefruit, honey dew, jicama, kiwifruit, lettuce, leeks, lemon, lime, Loblolly pine, maize, mango, melon, mushroom, nectarine, nut, oat, okra, onion, orange, an ornamental plant, papaya, parsley, pea, peach, peanut, pear, pepper, persimmon, pine, pineapple, plantain, plum, pomegranate, poplar, potato, pumpkin, quince, radiata pine, radicchio, radish, rapeseed, raspberry, rice, rye, rye grass, scallion, sorghum, Southern pine, soybean, spinach, squash, strawberry, sugar beet, sugarcane, sunflower, sweet potato, sweetgum, switchgrass, tangerine, tea, tobacco, tomato, turf, turnip, a vine, watermelon, wheat, yams, and zucchini.

16. A method for producing Methionine in a transgenic plant comprising growing the transgenic plant of claim 12 under suitable conditions for the production of Methionine, wherein the transgenic plant has an increased level of Methionine compared to a plant not comprising the expression units.

17. A method for preparing a transgenic plant that produces Methionine which comprises regenerating a transgenic plant from the plant cell of claim 1, wherein the transgenic plant has an increased level of Methionine compared to a plant not comprising the expression units.

18. A method of producing a crop of transgenic plants having an increased level of Methionine comprising multiplying the transgenic seed of claim 12 or breeding plants grown from said transgenic seed obtain a crop of transgenic plants having an increased level of Methionine.

19. Feed comprising the transgenic plant of claim 12, a plant organ of said transgenic plant, seed of said transgenic plant or an extract of said transgenic plant.

20. The feed of claim 19, wherein the feed is an animal fee or an aqua feed.


SEQUENCE LISTING (11 sequences)

SEQ ID NO: 1 - DNA, 603 bp, Bos taurus
atggagcggaccgaggtgctaaagccccgcaccctggccgatctgatccgcgtcctgcaccagctcttcgccggcgaggagatcaacgtggaggaagtgcaggccgtcatggaagcctatgagagcaaccccgccgagtgggcagtgtacgccaagttcgaccagtacaggtatactcgaaatcttgtggatcaaggaaatggaaagtttaatctcatgattctatgctggggtgaaggacatggcagcagtatccatgatcacaccgactcccactgctttctgaagatgctgcagggaaatctaaaggagacattgtttgcctggcctgacaagaaatccaatgagatgatcaagaagtctgaaagaatcttgagggaaaaccagtgtgcctacatcaatgattccattggcttacatcgagtagagaatattagccatacagagcctgccgtgagccttcacttgtatagtccgccttttgacacatgccacgcctttgatcaaagaacaggacataaaaacaaagtcatcatgacattccatagcaaatttggaatcaagactccatttacaacttcaggatccctggagaacaactaa

SEQ ID NO: 2 - DNA, 606 bp, Danio rerio
atggagcagactgaagtcatgaagcccgagactctggaggatctgatcaaaactctgcatcagatcttccagagcgactccatcaatgtggaggaggtgcagaacctgatggagtcctaccagagcaacccgcaggactggatgaagttcgccaagttcgaccagtacaggtacaccaggaacctcgtggatgaaggaaacggaaagttcaacctgatgatcctgtgctggggtgaaggacacggcagcagcatccatgaccacacagactcgcactgcttcctgaagctgctgcagggtcagctgaaggagacgctgttcgactggcccgaccgcaagctgcagagcggcatgaagccccgcggccagagcgtgctgcaggagaaccagtgcgcgtacatcaacgactctctgggactccaccgtgtggagaatgtgagccacacagagccggccgtgagtctgcacctttacagtcctccgttccagagctgccgcacgtttgaccagcgcaccggacaccacaacaccgtcaagatgaccttctggagcaaatatggcgagaggacgccctatgagctgagcgtctcgcaggagaataactga

SEQ ID NO: 3 - protein, 200 aa, Bos taurus
MERTEVLKPRTLADLIRVLHQLFAGEEINVEEVQAVMEAYESNPAEWAVYAKFDQYRYTRNLVDQGNGKFNLMILCWGEGHGSSIHDHTDSHCFLKMLQGNLKETLFAWPDKKSNEMIKKSERILRENQCAYINDSIGLHRVENISHTEPAVSLHLYSPPFDTCHAFDQRTGHKNKVIMTFHSKFGIKTPFTTSGSLENN

SEQ ID NO: 4 - protein, 201 aa, Danio rerio
MEQTEVMKPETLEDLIKTLHQIFQSDSINVEEVQNLMESYQSNPQDWMKFAKFDQYRYTRNLVDEGNGKFNLMILCWGEGHGSSIHDHTDSHCFLKLLQGQLKETLFDWPDRKLQSGMKPRGQSVLQENQCAYINDSLGLHRVENVSHTEPAVSLHLYSPPFQSCRTFDQRTGHHNTVKMTFWSKYGERTPYELSVSQENN

SEQ ID NO: 5 - DNA, 1482 bp, Equus caballus
atggctgactctgaaccgctcctctcccttgatggggaccccgtggctgcagaagccttgctccgggatgtgtttgggatcattgtggatgaggtcattcggaaagggaccagtgcctccgagaaggtctgcgagtggaaggagccggaggagctgaagcagctgctggatttggagctgcggagccatggggagtcacgggagcagatcctggagcggtgccgggctgtcatccgctacagcgtgaagacctgtcaccctcacttcttcaaccagctcttctcagggttggatccccacgctctggccgggcgcattgtcaccgagagccttaacaccagccagtacacttatgaaatcgcccccgtgtttgtgctcatggaagaagaggtcctgaagaaactccgggcgctggtgggctggagctctggcgatggggtcttctgccctggtggctccatctccaacatgtatgctgtgaacctggcccgctatcagcgctacccggattgcaagcagaggggcctccgggcactgccgcccctggccctcttcacatcgaaggagtgtcattactccatcaagaagggagctgcttttctgggacttggcactgacagtgtccgagtggtcaaggcagatgagagagggaaaatgatccctgaggatctggagaggcagatcagtctggccgaggcggagggtgctgtgccattcctggtcactgccacctctggcacgaccgtgctgggggcctttgatcccctggaggcgattgctgatgtgtgccagcgtcatgggctgtggctgcatgtggacgccgcctggggtgggagtgtcctgctctcacagacacacagacatctcctggctgggatccagagggcggactccgtggcctggaatccccacaagctcctcacagcaggcctgcagtgctcagctctcctgctccgggatacctcgaacctgctcaagcgctgccacgggtcccaggccagctacctcttccagcaggacaagttctacgacgtggctctggacacaggagacaaggtggtgcagtgcggccgccgcgtggactgtctgaagctgtggctcatgtggaaggcccagggcgggcaagggctggagcagcgagtggaccaggccttcgcccttgcccggtacctggtggaggaattgaagaagcgggaaggatttgagttggttatggagcctgagtttgtcaacgtgtgtttctggttcgtcccgcccagcctgcgggggaaacaggggagtccagattatgctgaaaggcttgccaaggtggccccggtacttaaagagcgcatggtgaaggagggctccatgatggttggctaccagccccacgggacccggggcaactttttccgcatggttgtggccaacccggctctgacccaggctgatatggacttcttcctcaatgagctggaacggctaggccaggacctctga

SEQ ID NO: 6 - DNA, 1449 bp, Danio rerio
atggacgagtctgatgggaagctgttccttactgaggctttcaacataatcatggaagaaattcttaacaaaggaagggacttgaaggagaaggtttgtgagtggaaagatccagatcagctgagatctctcctggacctcgaacttcgggatcatggagaatgtcatgagaagctgctgcagagggttcgagatgtggccaaatacagcgtaaaaacttgtcatcctcggttcttcaatcagctgtttgctggcgtggactatcatgcactgacaggacggctcatcactgaaaccctcaataccagccaatacacctatgaagtggctccagtgtttgtcctgatggaggaggaagtgatcagtaagcttcgctctctggttggctggtcagaaggagatgggatcttttgtcctggaggatccatgtctaacatgtatgccattaacgtcgctcggtactgggcttttcctcaagtgaagacaaaaggcttgtgggccgcaccacggatggctatatttacatcacaacagagtcattactccgtgaaaaaaggagctgcgtttcttggtattggaacagaaaatgttttcattgtgcaagtggatgagagcggcagcatgataccagaagacctggaggcaaaaattgtgcaggcaaaatcccaagacgctgttccgtttttcgtaaacgccacagccggaaccacagtgcagggagcctttgaccctctgaagcgcatagctgacatatgtgaaagaaacggcatgtggatgcatgttgacgccgcatggggaggaagcgtgctgttttccaaaaagcacagacatctggttgcaggaatagaaagagcaaactcggtgacttggaatcctcacaaaatgcttctgacgggactgcagtgctctgtgattttgttcagagatactacgaatttgctcatgcactgtcacagtgccaaagccacatacttgttccagcaagacaagttctacgacacaagtctggacacgggcgacaaatccatccagtgtggccggaaggtggattgcctcaagctctggctcatgtggaaggcaatcggagctagtggtctttcacagcgtgtcgataaggcctttgccctcactaggtatttagttgaagaaatggagaaacgggagaatttccagctggtctgtaaggggccgtttgtgaacgtttgcttctggtttattccacccagtctgaaaggaaaggagaacagcccagattaccaggaaagactatccaaggtggcgccagtcattaaagagaggatgatgaagcgaggaacgatgatggtgggatatcagccaatggatgaacacgtcaacttcttccgcatggtggttgtttctccacagctcacaaccaaagacatggatttcttccttgatgagatggagaaactcgggaaggatctatga

SEQ ID NO: 7 - protein, 493 aa, Equus caballus
MADSEPLLSLDGDPVAAEALLRDVFGIIVDEVIRKGTSASEKVCEWKEPEELKQLLDLELRSHGESREQILERCRAVIRYSVKTCHPHFFNQLFSGLDPHALAGRIVTESLNTSQYTYEIAPVFVLMEEEVLKKLRALVGWSSGDGVFCPGGSISNMYAVNLARYQRYPDCKQRGLRALPPLALFTSKECHYSIKKGAAFLGLGTDSVRVVKADERGKMIPEDLERQISLAEAEGAVPFLVTATSGTTVLGAFDPLEAIADVCQRHGLWLHVDAAWGGSVLLSQTHRHLLAGIQRADSVAWNPHKLLTAGLQCSALLLRDTSNLLKRCHGSQASYLFQQDKFYDVALDTGDKVVQCGRRVDCLKLWLMWKAQGGQGLEQRVDQAFALARYLVEELKKREGFELVMEPEFVNVCFWFVPPSLRGKQGSPDYAERLAKVAPVLKERMVKEGSMMVGYQPHGTRGNFFRMVVANPALTQADMDFFLNELERLGQDL

SEQ ID NO: 8 - protein, 482 aa, Danio rerio
MDESDGKLFLTEAFNIIMEEILNKGRDLKEKVCEWKDPDQLRSLLDLELRDHGECHEKLLQRVRDVAKYSVKTCHPRFFNQLFAGVDYHALTGRLITETLNTSQYTYEVAPVFVLMEEEVISKLRSLVGWSEGDGIFCPGGSMSNMYAINVARYWAFPQVKTKGLWAAPRMAIFTSQQSHYSVKKGAAFLGIGTENVFIVQVDESGSMIPEDLEAKIVQAKSQDAVPFFVNATAGTTVQGAFDPLKRIADICERNGMWMHVDAAWGGSVLFSKKHRHLVAGIERANSVTWNPHKMLLTGLQCSVILFRDTTNLLMHCHSAKATYLFQQDKFYDTSLDTGDKSIQCGRKVDCLKLWLMWKAIGASGLSQRVDKAFALTRYLVEEMEKRENFQLVCKGPFVNVCFWFIPPSLKGKENSPDYQERLSKVAPVIKERMMKRGTMMVGYQPMDEHVNFFRMVVVSPQLTTKDMDFFLDEMEKLGKDL

SEQ ID NO: 9 - DNA, 177 bp, Arabidopsis thaliana
atggctgcttatggtcaaatctcctcgggaatgactgtagatcctcaggttctctcttcctccagaaacattggagtttccctatcacctctccggagaacactaatcggcgccggagttaggtctactagtatctctctccgtcaatgttctctctccgttagatcgattaaaatc

SEQ ID NO: 10 - protein, 59 aa, Arabidopsis thaliana
MAAYGQISSGMTVDPQVLSSSRNIGVSLSPLRRTLIGAGVRSTSISLRQCSLSVRSIKI

SEQ ID NO: 11 - DNA, 39 bp, Artificial Sequence
  Synthetic Construct
agtactgaaggcgaagttaacgcggaagaagaaggcttt